(19) United States
(12) Patent Application Publication
(10) Pub. No.: US 2022/0349006 A1
(43) Pub. Date: Nov. 3, 2022

(54) CAP GUIDES AND METHODS OF USE THEREOF FOR RNA MAPPING

(71) Applicant: Moderna TX, Inc., Cambridge, MA (US)

(72) Inventors: Nicholas J. Amato, Cambridge, MA (US); Serenus Hua, Cambridge, MA (US); Kerry Salandria, Cambridge, MA (US)

(73) Assignee: Moderna TX, Inc., Cambridge, MA (US)

(21) Appl. No.: 17/761,420

(22) PCT Filed: Sep. 18, 2020
(86) PCT No.: PCT/US2020/051583

§ 371 Date: Mar. 17, 2022

Related U.S. Application Data
(60) Provisional application No. 62/902,604, filed on Sep. 19, 2019.

(51) Int. Cl. C12Q 1/6876; C12Q 1/6816; C12Q 1/44

(52) U.S. Cl. CPC C12Q 1/6876; C12Q 1/6816; C12Q 1/44

(57) ABSTRACT

The present disclosure relates, in some embodiments, to isolated nucleic acids (also referred to as cap guides) and methods of use thereof for RNA mapping. The disclosure is based, in part, on guide RNAs that bind to a position that is at least 7 nucleotides downstream of the first nucleotide of an mRNA molecule.

### RELATED APPLICATIONS

This Application claims the benefit under 35 U.S.C. 119(e) of the filing date of U.S. provisional Application Ser. No. 62/902,604, filed Sep. 19, 2019, entitled “CAP GUIDES AND METHODS OF USE THEREOF FOR RNA MAPPING”, the entire contents of which are incorporated by reference herein.

### FIELD

The invention relates to methods for the characterization of messenger RNA (mRNA) during the mRNA production process.

### BACKGROUND

Confirmation of structural variants of large mRNA such as sequence aborts, heterogeneous polyA tails, or folded structures is necessary for characterization of manufactured mRNA-based products for preclinical and clinical studies to ensure consistency, safety, and activity of the preparations. The large size and structural variants impose a challenge for many of the available analytical tools that do not have the required resolution or sensitivity.

### SUMMARY

The present disclosure is based, at least in part, on the design, screening, and selection of Cap guides that are useful for measuring the relative abundance of certain nucleic acid species (e.g., Cap species, coding region species, polyA tail species, etc.) on mRNA after treatment with RNase H and phosphatase. The disclosure is based, in part, on isolated nucleic acids that specifically bind (e.g., hybridize) to a target nucleic acid, such as an mRNA molecule, at a position that is at least 7 nucleotides downstream of (e.g., 3′ relative to) the first nucleic acid residue of the target nucleic acid. In some aspects, such isolated nucleic acids comprise one or more modifications, for example one or more 2′-O-methyl (2′OMe) modifications, one or more phosphorothioate (PS) modifications, or a combination thereof. In some embodiments, isolated nucleic acids (e.g., Cap guides) described herein detect mRNA species with higher sensitivity and/or specificity relative to previously described guide nucleic acids.

Accordingly, aspects of the disclosure relate to an isolated nucleic acid represented by the formula from 5′ to 3′:

[R]_{q}D₁D₂D₃D₄[R]ₚ

wherein each R is an unmodified or modified RNA base, D is a deoxyribonucleotide base and each of q and p are independently an integer between 0 and 50, wherein the isolated nucleic acid hybridizes to an mRNA at a position that is at least 7 nucleotides downstream of the first nucleotide of the mRNA, and wherein hybridization of the isolated nucleic acid to the mRNA in the presence of RNase H results in cleavage of the mRNA by the RNase H. In some embodiments, the mRNA comprises a 5′ UTR set forth in SEQ ID NO: 1 or SEQ ID NO: 2. In some embodiments, D₁ and D₃ comprise cytosine (C), and D₂ and D₄ comprise thymine (T). In some embodiments, each R comprises a 2′OMe modification and/or a phosphorothioate modification.

Aspects of the disclosure relate to an isolated nucleic acid represented by the formula from 5′ to 3′:

[R]_{q}D₁D₂D₃D₄[R]ₚ

wherein each R is an unmodified or modified RNA base, D is a deoxyribonucleotide base and each of q and p are independently an integer between 0 and 50, wherein hybridization of the isolated nucleic acid to a mRNA 5′ untranslated region (5′ UTR) at a +7 position in the presence of RNase H results in cleavage of the mRNA 5′ UTR by the RNase H, and wherein the mRNA 5′ UTR comprises SEQ ID NO: 1 or SEQ ID NO: 2. In some embodiments, D₁ and D₃ comprise cytosine (C), and D₂ and D₄ comprise thymine (T).

Aspects of the disclosure relate to an isolated nucleic acid represented by the formula from 5′ to 3′:

[R]_{q}D₁D₂D₃D₄[R]ₚ

wherein each R is an unmodified or modified RNA base, D is a deoxyribonucleotide base and each of q and p are independently an integer between 0 and 50, wherein D₁ and D₃ comprise cytosine (C), and D₂ and D₄ comprise thymine (T), and wherein hybridization of the isolated nucleic acid to a mRNA 5′ untranslated region (5′ UTR) at a +7 position in the presence of RNase H results in cleavage of the mRNA 5′ UTR by the RNase H.

In some embodiments, at least one R is a modified RNA nucleotide, optionally a 2′-O-methyl modified RNA nucleotide, a 2′-fluoro modified RNA nucleotide, a peptide nucleic acid (PNA), or a locked nucleic acid (LNA). In some embodiments, at least one R comprises a modified RNA backbone, optionally a phosphorothioate (PS) backbone. In some embodiments, at least one of D₁, D₂ D₃, and D₄ are unmodified deoxyribonucleotide bases. In some embodiments, at least one of D₁, D₂ D₃, and D₄ are modified deoxyribonucleotide bases.

In some embodiments, the modified deoxyribonucleotide base is 5-nitroindole, Inosine, 4-nitroindole, 6-nitroindole, 3-nitropyrrole, a 2-6-diaminopurine, 2-amino-adenine, or 2-thio-thiamine.

In some embodiments, the cleavage of the mRNA by the RNase H results in liberation of the 5′ UTR of the mRNA. In some embodiments, cleavage of the mRNA by the RNase H results in liberation of the polyA tail of the mRNA. In some embodiments, the cleavage of the mRNA (e.g., the mRNA 5′ UTR) by the RNase H results in liberation of an intact mRNA Cap. In some embodiments, the mRNA is in vitro transcribed (IVT) RNA.

In some embodiments, the isolated nucleic acid is selected from the sequences set forth in Table 2. In some embodiments, the isolated nucleic acid is SEQ ID NO: 3 or SEQ ID NO: 4. In some embodiments, the isolated nucleic acid is SEQ ID NO: 5 or SEQ ID NO: 6. In some embodiments, the isolated nucleic acid is SEQ ID NO: 7 or SEQ ID NO: 8. In some embodiments, the isolated nucleic acid is SEQ ID NO: 9 or SEQ ID NO: 10. In some embodiments, the isolated nucleic acid is SEQ ID NO: 11 or SEQ ID NO: 12. In some embodiments, the isolated nucleic acid is SEQ ID NO: 13 or SEQ ID NO: 14. In some embodiments, the isolated nucleic acid is SEQ ID NO: 15.

Aspects of the present disclosure relate to a composition comprising a plurality of isolated nucleic acids, wherein each of the isolated nucleic acids individually is an isolated nucleic acid as described herein. In some embodiments, the plurality is three or more isolated nucleic acids. In some embodiments, the composition further comprises a buffer, and optionally, RNase H enzyme.

Aspects of the present disclosure relate to a method of selecting an isolated nucleic acid, the method comprising: digesting a mRNA hybridized to an isolated nucleic acid provided herein with an RNase enzyme to produce a plurality of mRNA fragments; physically separating the plurality of mRNA fragments; generating a signature profile of the mRNA by detecting the plurality of mRNA fragments; comparing the signature profile with a known mRNA signature profile, and selecting the isolated nucleic acid based on the comparison of the signature profile with the known RNA signature profile.

In some embodiments, the selecting and/or the detecting comprises a method selected from the group consisting of gel electrophoresis, capillary electrophoresis, high pressure liquid chromatography (HPLC), and mass spectrometry. In some embodiments, the HPLC is HPLC-UV. In some embodiments, the mass spectrometry is Electrospray Ionization mass spectrometry (ESI-MS) or Matrix-assisted Laser Desorption/Ionization-Time of Flight (MALDI-TOF) mass spectrometry.

In some embodiments, the mRNA is mixed with a buffer comprising at least one component selected from the group consisting of urea, EDTA, magnesium chloride (MgCl₂) and Tris prior to digestion. In some embodiments, the mRNA and the buffer are incubated at a temperature between 60° C. to 100° C.

In some embodiments, methods provided herein further comprise incubating the mRNA sample with 2′,3′-Cyclic-nucleotide 3′-phosphodiesterase (CNP) following the digestion to produce a CNP treated mRNA sample. In some embodiments, the incubating of the mRNA with CNP is performed for about 1 hour. In some embodiments, methods further comprise incubating the CNP treated mRNA with Calf Intestinal Alkaline Phosphatase (CIP).

In some embodiments, methods further comprise incubating the mRNA with an enzymatic inhibitor. In some embodiments, the enzymatic inhibitor is EDTA.

In some embodiments, the signature profile is in the form of an absorbance spectrum or a mass spectrum.

In some embodiments, the isolated nucleic acid is an isolated nucleic acid described herein. In some embodiments, the mRNA 5′ untranslated region (5′ UTR) comprises SEQ ID NO: 1 or SEQ ID NO: 2.

In some embodiments, the signature profile comprises determining Cap structure of the mRNA based upon comparison of the signature profile with the known RNA signature profile.

Aspects of the present disclosure relate to a method for quality control of an RNA pharmaceutical composition, comprising digesting the RNA pharmaceutical composition with an RNase H enzyme to produce a plurality of RNA fragments; physically separating the plurality of RNA fragments; generating a signature profile of the RNA pharmaceutical composition by detecting the plurality of fragments; comparing the signature profile with a known RNA signature profile; and determining the quality of the RNA based on the comparison of the signature profile with the known RNA signature profile; wherein the digesting step comprises contacting the RNA pharmaceutical composition with an isolated nucleic acid described herein, or a pharmaceutical composition described herein prior to contacting the RNA pharmaceutical composition with an RNase H enzyme.

In some embodiments, the digestion step is performed in the presence of a blocking oligonucleotide. In some embodiments, the blocking oligonucleotide comprises at least one modified nucleotide, optionally wherein the modification is selected from locked nucleic acid nucleotide (LNA), 2′ OMe-modified nucleotide, and peptide nucleic acid (PNA) nucleotide. In some embodiments, the blocking oligonucleotide comprises one or more modified backbone linkages, for example one or more phosphorothioate linkages. In some embodiments, a blocking oligonucleotide comprises a completely modified backbone, for example a phosphorothioate (PS) backbone. In some embodiments, the blocking oligonucleotide targets the 5′ untranslated region (5′UTR), open reading frame, or the 3′ untranslated region (3′UTR) of the test mRNA.

In some embodiments, the mRNA is prepared by in vitro transcription (IVT). In some embodiments, the RNA is a therapeutic mRNA.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations of thereof herein, is meant to encompass the items listed thereafter and equivalents thereof as well as additional items.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 shows representative data of relative abundance of Cap species on mRNA after treatment with RNase H and phosphatase.

FIG. 2 shows representative mass spectrometry profiles of Cap species on mRNA after treatment with RNase H and phosphatase.

FIG. 3 shows representative total ion chromatogram (TIC) data for retention time of Cap species.

FIG. 4 shows representative data of relative Cap quantification comparison for Sample 1.

FIG. 5 shows representative data of relative Cap quantification comparison for Sample 6.

FIG. 6 shows representative structures of backbone modifications of interest.

FIG. 7 shows Cap guide sequences comprising flanking LNA or flanking LNA/2′OMe sequences. SEQ ID NOs: 18-23 are shown.

FIG. 8 shows representative data of normalized Cap1 abundance from backbone modification screening.

FIG. 9 shows representative total ion chromatogram (TIC) data for retention time from backbone modification screening.

FIG. 10 shows representative data of Cap1 abundance from analysis using modified Cap guides at different concentrations.

FIG. 11 shows representative data of Cap guide 7PS linearity.

FIG. 12A shows representative data of percent abundance of Cap guide 7PS and current Cap guide for Vaccinia capped mRNA.

FIG. 12B shows representative data of abundance of Cap guide 7PS and current Cap guide.

FIG. 13 shows representative data of percent abundance of Cap guide 7PS and current Cap guide for Vaccinia capped mRNA and Co-transcriptional (co-transcript) capped mRNA.

FIG. 14 shows representative data of co-transcript assay from LC-UV analysis. FIG. 15 shows representative data of effect of sequence variants from co-transcript LC-UV cap assay.

FIG. 16 shows representative data of previously described guide conditions versus peak area (top panel) and Int guide conditions versus peak area (bottom panel).

FIG. 17 shows representative data of guide concentration and digestion time for previously described guide and Int guide.

### DETAILED DESCRIPTION OF THE INVENTION

Delivery of mRNA molecules to a subject in a therapeutic context is promising because it enables intracellular translation of the mRNA and production of at least one encoded peptide or polypeptide of interest without the need for nucleic acid-based delivery systems (e.g., viral vectors and DNA-based plasmids). Therapeutic mRNA molecules are generally synthesized in a laboratory (e.g., by in vitro transcription). However, there is a potential risk of carrying over impurities or contaminants, such as incorrectly synthesized mRNA and/or undesirable synthesis reagents, into the final therapeutic preparation during the production process. In order to prevent the administration of impure or contaminated mRNA, the mRNA molecules can be subject to a quality control (QC) procedure (e.g., validated or identified) prior to use. Validation confirms that the correct mRNA molecule has been synthesized and is pure.

Provided herein are compositions and methods for analyzing and characterizing mRNA (e.g., target mRNA in a RNA sample). The disclosure is based, in part, on isolated nucleic acids that specifically bind (e.g., hybridize) to a target nucleic acid, such as an mRNA molecule, at a position that is at least 7 nucleotides downstream of (e.g., 3′ relative to) the first nucleic acid position of the target nucleic acid. In some embodiments, such isolated nucleic acids are referred to as “+7 guides” or “7 nt” guides. In some aspects, such isolated nucleic acids (e.g., 7 nt guides) comprise one or more modifications, for example one or more 2′-O-methyl (2′OMe) modifications, one or more phosphorothioate (PS) modifications, or a combination thereof. In some embodiments, isolated nucleic acids (e.g., Cap guides) described herein detect mRNA species with higher sensitivity and/or specificity relative to previously described guide nucleic acids.

In some embodiments, isolated nucleic acids of the present disclosure are used for analyzing and characterizing mRNA. Thus, in some embodiments, the present disclosure provides methods of selecting isolated nucleic acids for analyzing and characterizing mRNA. In some embodiments, the present disclosure provides methods for quality control of a mRNA pharmaceutical composition comprising isolated nucleic acids described herein.

### Isolated Nucleic Acids

In some aspects, the disclosure provides isolated nucleic acids (e.g., specific oligos) that anneal to a mRNA (e.g., a target mRNA) and direct RNase H cleavage of the mRNA. In some embodiments, the isolated nucleic acids are referred to as “guide strands” or “Cap guides”.

A “polynucleotide” or “nucleic acid” is at least two nucleotides covalently linked together, and in some instances, may contain phosphodiester bonds (e.g., a phosphodiester “backbone”) or modified bonds (e.g., a modified backbone), such as phosphorothioate bonds (e.g., a phosphorothioate (PS) backbone). An “isolated nucleic acid” is a nucleic acid that does not occur in nature. In some instances, mRNA in a mRNA sample comprises isolated mRNA. It should be understood, however, that while an isolated nucleic acid as a whole is not naturally-occurring, it may include nucleotide sequences that occur in nature. Thus, a “polynucleotide” or “nucleic acid” sequence is a series of nucleotide bases (also called “nucleotides”), generally in DNA and RNA, and means any chain of two or more nucleotides. The terms include genomic DNA, cDNA, RNA, any synthetic and genetically manipulated polynucleotide. This includes single- and double-stranded molecules; i.e., DNA-DNA, DNA-RNA, and RNA-RNA hybrids as well as “protein nucleic acids” (PNA) formed by conjugating bases to an amino acid backbone and “locked nucleic acids” formed by modifying the ribose moiety of an RNA with an extra bridge connecting the 2′ oxygen and 4′ carbon.

An isolated nucleic acid may range in length, for example from about 2 nucleotides in length to about 50,000 nucleotides in length. In some embodiments, an isolated nucleic acid ranges from about 2 to 10, 5 to 20, 10 to 50, 50 to 200, 100 to 500, 250 to 1000, 500 to 2500, 1000 to 5000, 2500, to 10,000, 5,000 to 25,000, 10,000 to 50,000, or more nucleotides in length. In some embodiments, an isolated nucleic acid is longer than 50,000 nucleotides in length.

Aspects of the disclosure relate to isolated nucleic acids (e.g., guides) that bind to a position of an mRNA that is at least 7 nucleotides “downstream” of the first nucleic acid position (e.g., nucleic acid base). In some embodiments, an isolated nucleic acid (e.g., guide) binds to an mRNA at a position that is 4, 5, 6, 7, 8, 9, 10, 11, 12, 13, 14, 15, 16, 17, 18, 19, 20, 21, 22, 23, 24, 25, or more nucleotides downstream of the first nucleic acid position. In some embodiments, an isolated nucleic acid (e.g., guide) binds to an mRNA at a position that is more than 25 nucleotides (e.g., 30, 40, 50, 100, 200, 500, or more) nucleotide downstream of the first nucleic acid position. In some embodiments, an isolated nucleic acid (e.g., guide) binds to one or more nucleic acid positions of an mRNA untranslated region (UTR), such as a 5′UTR or 3′UTR. In some embodiments, an isolated nucleic acid (e.g., guide) binds to one or more nucleic acid positions of a protein coding region of an mRNA (e.g., one or more positions between a 5′ UTR and a 3′UTR of an mRNA, such as an open reading frame). In some embodiments, an isolated nucleic acid binds about 1, 2, 3, 4, 5, 6, 7, 8, 9, 10, 11, 12, 13, 14, 15, 16, 17, 18, 19, 20, 21, 22, 23, 24, 25, or more nucleotides upstream of the last protein coding nucleic acid position (e.g., the last nucleic acid position of a “stop” codon).

In some aspects, the disclosure relates to an isolated nucleic acid represented by the formula from 5′ to 3′:

[R]_{q}D₁D₂D₃D₄[R]ₚ

wherein each R is an unmodified or modified RNA base, D is a deoxyribonucleotide base and each of q and p are independently an integer between 0 and 50, wherein the isolated nucleic acid hybridizes to an mRNA at a position that is at least 7 nucleotides downstream of the first nucleotide of the mRNA, wherein hybridization of the isolated nucleic acid to the mRNA in the presence of RNase H results in cleavage of the mRNA by the RNase H. In some embodiments, the mRNA comprises a 5′ UTR set forth in SEQ ID NO: 1 or SEQ ID NO: 2.

In some aspects, the disclosure provides an isolated nucleic acid represented by the formula from 5′ to 3′:

[R]_{q}D₁D₂D₃D₄[R]ₚ

wherein each R is a modified or unmodified RNA base, D is a deoxyribonucleotide base, and each of q and p are independently an integer between 0 and 50, wherein hybridization of the isolated nucleic acid to a nucleic acid position that is at least 7 nt into an mRNA 5′ untranslated region (5′ UTR) in the presence of RNase H results in cleavage of the mRNA 5′ UTR by the RNase H. In some embodiments, the mRNA 5′ UTR comprises SEQ ID NO: 1 or SEQ ID NO: 2.

TABLE 1
mRNA sequences.

| | SEQ ID |
|---|---|
| Sequence | NO: |
| GGGAAATAAGAGAGAAAAGAAGAGTAA | 1 |
| GAAGAAATATAAGAGCCACC | |
| GGGAAATAAGAGAGAAAAGAAGAGTAAG | 2 |
| AAGAAATATAAGACCCCGGCGCCGCCACC | |

In some aspects, the disclosure provides an isolated nucleic acid represented by the formula from 5′ to 3′:

[R]_{q}D₁D₂D₃D₄[R]ₚ

wherein each R is a modified or unmodified RNA base, D is a deoxyribonucleotide base, and each of q and p are independently an integer between 0 and 50, wherein D₁ and D₃ comprise cytosine (C), and D₂ and D₄ comprise thymine (T), and wherein hybridization of the isolated nucleic acid to a mRNA 5′ untranslated region (5′ UTR) in the presence of RNase H results in cleavage of the mRNA 5′ UTR by the RNase H.

In some embodiments, at least one R is a modified RNA nucleotide, for example a 2′-O-methyl modified RNA nuceleotide. Examples of modifications include, but are not limited to pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, 2′-O-methyl uridine, and 2′-Fluoro. Other examples of modifications useful in the mRNA described herein include those listed in US patent application publication number 2015/0064235.

In some embodiments, at least one R comprises a backbone modification. A “backbone modification” refers to incorporation of one or more non-naturally occurring phosphate-based bonds in an isolated nucleic acid. For example, the phosphate group of the nucleotide may be modified, e.g., by substituting one or more of the oxygens of the phosphate group with sulfur (e.g., phosphorothioates (PS)), or by making other substitutions which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) preferably decrease the rate of hydrolysis of, for example, polynucleotides comprising said analogs in vivo or in vitro. In some embodiments, each R of an isolated nucleic acid comprises a backbone modification (e.g., the guide comprises a completely modified backbone with respect to the “R” portions).

The length of each of [R]_{q} and [R]ₚ can independently vary in length. For example, in some embodiments, q is an integer between 0 and 50 (e.g., 0, 1, 2, 3, 4, 5, 6, 7, 8, 9, 10, 11 ,12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) and p is an integer between 0 and 50 (e.g., 0, 1, 2, 3, 4, 5, 6, 7, 8, 9, 10, 11 ,12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50).

In some embodiments, q is an integer between 0 and 30 (e.g., 0, 1, 2, 3, 4, 5, 6, 7, 8, 9, 10, 11 ,12, 13, 14, 15, 16, 17, 18, 19, 20, 21, 22, 23, 24, 25, 26, 27, 28, 29, or 30) and p is an integer between 0 and 50 (e.g., 0, 1, 2, 3, 4, 5, 6, 7, 8, 9, 10, 11 ,12, 13, 14, 15, 16, 17, 18, 19, 20, 21, 22, 23, 24, 25, 26, 27, 28, 29, or 30).

In some embodiments, q is an integer between 0 and 15 (e.g., 0, 1, 2, 3, 4, 5, 6, 7, 8, 9, 10, 11 ,12, 13, 14, or 15) and p is an integer between 0 and 15 (e.g., 0, 1, 2, 3, 4, 5, 6, 7, 8, 9, 10, 11 ,12, 13, 14, or 15).

In some embodiments, q is an integer between 0 and 6 (e.g., 0, 1, 2, 3, 4, 5, or 6) and p is an integer between 1 and 10 (e.g., 1, 2, 3, 4, 5, 6, 7, 8, 9, or 10). In some embodiments, p is an integer between 0 and 6 (e.g., 0, 1, 2, 3, 4, 5, or 6) and q is an integer between 1 and 10 (e.g., 1, 2, 3, 4, 5, 6, 7, 8, 9, or 10).

In some embodiments, each of D₁ and D₂ are unmodified (e.g., natural) deoxyribonucleotide bases. As used herein, “unmodified deoxyribonucleotide base” refers to a natural DNA base, such as adenosine, guanosine, cytosine, thymine, or uracil. In some embodiments, D₃, D₄, or D₃ and D₄ are unnatural (e.g., modified) deoxyribonucleotide bases. In some embodiments, D₁ is an unnatural (e.g., modified) deoxyribonucleotide base. In some embodiments, D₂ is an unnatural (e.g., modified) deoxyribonucleotide base. In some embodiments, D₃ is an unnatural (e.g., modified) deoxyribonucleotide base. In some embodiments, D₄ is an unnatural (e.g., modified) deoxyribonucleotide base.

The term “modified deoxyribonucleotide base,” “nucleotide analog,” or “altered nucleotide” refers to a non-standard nucleotide, including non-naturally occurring deoxyribonucleotides. Preferred nucleotide analogs are modified at any position so as to alter certain chemical properties of the nucleotide yet retain the ability of the nucleotide analog to perform its intended function. Examples of positions of the nucleotide which may be derivitized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10(4):297-310.

Nucleotide analogs may also comprise modifications to the sugar portion of the nucleotides. For example the 2′ OH-group may be replaced by a group selected from H, OR, R, F, Cl, Br, I, SH, SR, NH₂, NHR, NR₂, COOR, or OR, wherein R is substituted or unsubstituted C₁-C.₆ alkyl, alkenyl, alkynyl, aryl, etc.

In some embodiments, the unnatural (e.g., modified) deoxyribonucleotide base is 5-nitroindole or Inosine. In some embodiments, the modified deoxyribonucleotide is 4-nitroindole, 6-nitroindole, 3-nitropyrrole, a 2-6-diaminopurine, 2-amino-adenine, or 2-thio-thiamine.

In some embodiments, hybridization of certain isolated nucleic acids (e.g., guide strands) to a mRNA in the presence of RNase H results in specific separation of mRNA 5′ untranslated region (5′ UTR) from the mRNA by the RNase H. Without wishing to be bound by any particular theory, separation of intact 5′UTR of an mRNA allows for characterization of the 5′ cap structure of the mRNA, for example by mass spectrometric analysis of the 5′ cap fragment. In some embodiments, isolated nucleic acids direct separation of intact 5′UTR of mRNA without digestion of other regions of the mRNA (e.g., open reading frame (ORF), 3′ untranslated region (UTR), polyA tail, etc.). In some embodiments, isolated nucleic acids direct separation of intact 5′UTR of mRNA and certain other portions of the mRNA (e.g., a coding sequence or portion thereof) without digestion of other regions of the mRNA (e.g., 3′ untranslated region (UTR), polyA tail, etc.).

In some embodiments, isolated nucleic acids (e.g., guide strands) that direct in RNase H cleavage of mRNA 5′ UTR can hybridize anywhere within the 5′ UTR region that is 7 or more nucleotides from the 5′ terminus of the mRNA (e.g. the region directly upstream of the first nucleotide of the mRNA initiation codon) of an mRNA. For example, in some embodiments, an isolated nucleic acid (e.g., guide strand) hybridizes to a mRNA 5′ UTR between 1 nucleotide and about 200 nucleotides upstream of the first nucleotide of the initiation codon. In some embodiments, an isolated nucleic acid (e.g., guide strand) hybridizes to a mRNA 5′ UTR between 1 nucleotide and about 100 nucleotides upstream of the first nucleotide of the initiation codon. In some embodiments, an isolated nucleic acid (e.g., guide strand) hybridizes to a mRNA 5′ UTR between 1 nucleotide and about 50 nucleotides (e.g., 1, 2, 3, 4, 5, 6, 7, 8, 9, 10, 11 ,12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides) upstream of the first nucleotide of the initiation codon. Non-limiting examples of isolated nucleic acids (e.g., guide strands) that result in RNase H cleavage of mRNA 5′UTR are shown in Table 2.

TABLE 2
Non-limiting examples of cap-targeting
RNase H guide stands.

| | SEQ | Guide |
|---|---|---|
| | ID | ID |
| Sequence | NO: | NO: |
| CCCUUUAUUCTCTUAC | 3 | Control |
| AUUCTCTCUUUU | 4 | 1 |
| AUUCTCTCUUUUC | 5 | 2 |
| AUUCTCTCUUUUCU | 6 | 3 |
| UUCTCTCUUUU | 7 | 4 |
| UCTCTCUUUU | 8 | 5 |
| CTCTCUUUU | 9 | 6 |
| AUUCTCTCUUUUCUUCUCAUUC | 10 | 7 |
| AUUCTCTCUUCCCUUCUCACCC | 11 | 7a |
| AUUCTCTCUCCCCUUC | 12 | 8 |
| AUUCTCTCUUUUCUUCUCAUUC | 13 | 9 |
| UUCUUUAUAUUC | | |
| AUUCTCTCUU | 14 | 10 |
| AUUCTCTUAC | 15 | 11 |

Compositions comprising a plurality of isolated nucleic acids (e.g., a cocktail of guide strands) are also contemplated by the disclosure. In some embodiments, compositions comprising a plurality of isolated nucleic acids (e.g., a cocktail of guide strands) are useful for the simultaneous (e.g., “one pot”) digestion, and subsequent separation, of various regions of an mRNA, including but not limited to 5′UTR, ORF, and 3′UTR. Compositions described by the disclosure may contain between 2 and 100 isolated nucleic acids (e.g., between 2 and 100 guide strands). In some embodiments, a composition comprising a plurality of guide strands comprises 2, 3, 4, 5, 6, 7, 8, 9, or 10 unique isolated nucleic acid (e.g., guide strands). In some embodiments, a composition comprises three different isolated nucleic acids (e.g., guide strands). For example, using one, or two guide strands at a time (e.g. serially), multiple orthogonal digests of an mRNA can be performed in parallel with the same procedure and run time, allowing for greater sequence coverage during RNase mapping.

In some aspects, the disclosure provides a composition comprising a plurality of isolated nucleic acids as described by the disclosure. In some embodiments, the plurality is three or more isolated nucleic acids. In some embodiments, the plurality is three or more isolated nucleic acids selected from the group consisting of SEQ ID NOs: 3-15.

In some embodiments, the plurality comprises between 5 and 50 isolated nucleic acids that each results in cleavage of a different portion of the mRNA (e.g., cleavage of the 5′UTR, open reading frame, 3′UTR, polyA tail, etc.). In some embodiments, the plurality comprises between 5 and 50 isolated nucleic acids that each results in cleavage of the mRNA 5′ UTR. In some embodiments, the plurality comprises between 10 and 20 isolated nucleic acids that each results in cleavage of a different portion of the mRNA (e.g., cleavage of the 5′UTR, open reading frame, 3′UTR, polyA tail, etc.). In some embodiments, the plurality comprises between 1 and 5 isolated nucleic acids that each results in cleavage of a different portion of the mRNA (e.g., cleavage of the 5′UTR, open reading frame, 3′UTR, polyA tail, etc.). In some embodiments, the plurality comprises between 10 and 20 isolated nucleic acids that each results in cleavage of the mRNA 5′ UTR. In some embodiments, the plurality comprises between 1 and 5 isolated nucleic acids that each results in cleavage of the mRNA 5′UTR.

In some embodiments, the plurality comprises: (i) at least one isolated nucleic acid that results in cleavage of the mRNA 5′UTR (e.g., an isolated nucleic acid provided herein), and (ii) at least one isolated nucleic acid that results in cleavage of the mRNA 3′UTR.

In some embodiments, the plurality comprises: (i) at least one isolated nucleic acid that results in cleavage of the mRNA 5′UTR (e.g., an isolated nucleic acid provided herein), (ii) at least one isolated nucleic acid that results in cleavage of the mRNA 3′UTR; and, (iii) at least one isolated nucleic acid that results in cleavage of the mRNA ORF.

Isolated nucleic acids (e.g., guide strands) that result in RNase H cleavage of mRNA 3′ UTR can hybridize anywhere within the 3′ UTR region (e.g. the region directly downstream of the last nucleotide of the mRNA stop codon) of an mRNA. For example, in some embodiments, an isolated nucleic acid (e.g., guide strand) hybridizes to a mRNA 3′ UTR between 1 nucleotide and about 200 nucleotides downstream of the last nucleotide of the stop codon. In some embodiments, an isolated nucleic acid (e.g., guide strand) hybridizes to a mRNA 3′ UTR between 1 nucleotide and about 100 nucleotides downstream of the last nucleotide of the stop codon. In some embodiments, an isolated nucleic acid (e.g., guide strand) hybridizes to a mRNA 3′ UTR between 1 nucleotide and about 50 nucleotides (e.g., 1, 2, 3, 4, 5, 6, 7, 8, 9, 10, 11 ,12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides) downstream of the last nucleotide of the stop codon.

In some embodiments, hybridization of the isolated nucleic acid to a mRNA in the presence of RNase H results in cleavage of the mRNA open reading frame (ORF) by the RNase H, and no cleavage of the 5′ UTR or 3′UTR of the mRNA. Without wishing to be bound by any particular theory, shortening the length of an isolated nucleic acid (e.g. guide strand) allows it to land in more places on the ORF, progressively reducing secondary structure leading to specific total digest of the mRNA. Accordingly, in some embodiments, an isolated nucleic acid (e.g., guide strand) that directs cleavage of a mRNA ORF is between 4 and 16 nucleotides in length (e.g., 4, 5, 6, 7, 8, 9, 10, 11, 12, 13, 14, 15, or 16 nucleotides in length). In some embodiments, a guide strand comprises a single 5′ or 3′ positioned 2′O-methyl RNA and four unmodified DNA bases. In some embodiments, a guide strand consists of four unmodified DNA bases.

In some embodiments, compositions described by the disclosure further comprise a buffer, and optionally, RNase H enzyme.

### Target RNA

Aspects of the invention relate to cap guides that anneal to target RNA (e.g., target mRNA). In some embodiments, a cap guide anneals to RNA in an RNA sample. RNA is composed of repeating ribonucleosides. It is possible that the RNA includes one or more deoxyribonucleosides. In preferred embodiments the RNA is comprised of greater than 60%, 70%, 80% or 90% of ribonucleosides. In other embodiments the RNA is 100% comprised of ribonucleosides. The RNA in an RNA sample is preferably an mRNA.

As used herein, the term “messenger RNA (mRNA)” refers to a ribonucleic acid that has been transcribed from a DNA sequence by an RNA polymerase enzyme, and interacts with a ribosome to synthesize protein encoded by DNA. Generally, mRNA are classified into two sub-classes: pre-mRNA and mature mRNA. Precursor mRNA (pre-mRNA) is mRNA that has been transcribed by RNA polymerase but has not undergone any post-transcriptional processing (e.g., 5′capping, splicing, editing, and polyadenylation). Mature mRNA has been modified via post-transcriptional processing (e.g., spliced to remove introns and polyadenylated region) and is capable of interacting with ribosomes to perform protein synthesis.

mRNA can be isolated from tissues or cells by a variety of methods. For example, a total RNA extraction can be performed on cells or a cell lysate and the resulting extracted total RNA can be purified (e.g., on a column comprising oligo-dT beads) to obtain extracted mRNA.

Alternatively, mRNA can be synthesized in a cell-free environment, for example by in vitro transcription (IVT). IVT is a process that permits template-directed synthesis of ribonucleic acid (RNA) (e.g., messenger RNA (mRNA)). It is based, generally, on the engineering of a template that includes a bacteriophage promoter sequence upstream of the sequence of interest, followed by transcription using a corresponding RNA polymerase. In vitro mRNA transcripts, for example, may be used as therapeutics in vivo to direct ribosomes to express protein therapeutics within targeted tissues.

Traditionally, the basic components of an mRNA molecule include at least a coding region, a 5′UTR, a 3′UTR, a 5′ cap and a poly-A tail. IVT mRNA may function as mRNA but are distinguished from wild-type mRNA in their functional and/or structural design features which serve to overcome existing problems of effective polypeptide production using nucleic-acid based therapeutics. For example, IVT mRNA may be structurally modified or chemically modified. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to affect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

An RNA may comprise naturally occurring nucleotides and/or non-naturally occurring nucleotides such as modified nucleotides. In some embodiments, the RNA polynucleotide of the RNA vaccine includes at least one chemical modification. In some embodiments, the chemical modification is selected from the group consisting of pseudouridine, N1-methylpseudouridine, 2-thiouridine, 4′-thiouridine, 5-methylcytosine, 2-thio-l-methyl-1-deaza-pseudouridine, 2-thio-1-methyl-pseudouridine, 2-thio-5-aza-uridine , 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine. Other exemplary chemical modifications useful in the mRNA described herein include those listed in US Published patent application 2015/0064235.

An “in vitro transcription template (IVT),” as used herein, refers to deoxyribonucleic acid (DNA) suitable for use in an IVT reaction for the production of messenger RNA (mRNA). In some embodiments, an IVT template encodes a 5′ untranslated region, contains an open reading frame, and encodes a 3′ untranslated region and a polyA tail. The particular nucleotide sequence composition and length of an IVT template will depend on the mRNA of interest encoded by the template.

A “5′ untranslated region (UTR)” refers to a region of an mRNA that is directly upstream (i.e., 5′) from the start codon (i.e., the first codon of an mRNA transcript translated by a ribosome) that does not encode a protein or peptide.

A “3′ untranslated region (UTR)” refers to a region of an mRNA that is directly downstream (i.e., 3′) from the stop codon (i.e., the codon of an mRNA transcript that signals a termination of translation) that does not encode a protein or peptide.

An “open reading frame” is a continuous stretch of DNA or RNA beginning with a start codon (e.g., methionine (ATG)), and ending with a stop codon (e.g., TAA, TAG or TGA) and encodes a protein or peptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation. However, in some embodiments, mRNA molecules do not comprise a polyA tail. In some embodiments, such molecules are referred to as “tailless”.

In some embodiments, the test or target mRNA (e.g., IVT mRNA) is a therapeutic mRNA. As used herein, the term “therapeutic mRNA” refers to an mRNA molecule (e.g., an IVT mRNA) that encodes a therapeutic protein. Therapeutic proteins mediate a variety of effects in a host cell or a subject in order to treat a disease or ameliorate the signs and symptoms of a disease. For example, a therapeutic protein can replace a protein that is deficient or abnormal, augment the function of an endogenous protein, provide a novel function to a cell (e.g., inhibit or activate an endogenous cellular activity, or act as a delivery agent for another therapeutic compound (e.g., an antibody-drug conjugate). Therapeutic mRNA may be useful for the treatment or prevention through vaccination for the following diseases and conditions: bacterial infections, viral infections, parasitic infections, cell proliferation disorders, genetic disorders, and autoimmune disorders.

A “test mRNA” or “target mRNA” (used interchangeably herein) is an mRNA of interest, having a known nucleic acid sequence. The target mRNA may be found in a RNA or mRNA sample. In addition to the target mRNA, the RNA or mRNA sample may include a plurality of mRNA molecules or other impurities obtained from a larger population of mRNA molecules. For example, after the production of IVT mRNA, a target mRNA sample may be removed from the population of IVT mRNA in order to assay for the purity and/or to confirm the identity of the mRNA produced by IVT.

Characterizing mRNA Species

Methods provided herein relate to characterizing mRNA using guides provided herein. In some embodiments, characterizing mRNA comprises digestion of a target mRNA to produce two or more fragments (e.g., portions or species, such as a Cap species, ORF species, 3′UTR species, etc.) of the mRNA that are characteristic of the mRNA. In some embodiments, characterizing mRNA comprises digestion of a target mRNA Cap. mRNA capping is a process by which the 5′end of the mRNA is modified with a 7-methylguanylate cap (also referred to as “Cap”) to create stable and mature messenger RNA able to undergo translation during protein synthesis. In certain cases, the mRNA capping process is incomplete, leaving mRNA having a partial Cap (e.g., Cap that is not methylated at position 7) or uncapped mRNA. In some embodiments, it is desirable to map the 5′ UTR of an mRNA to identify whether the mRNA contains Cap, partial Cap, or is uncapped (also referred to as relative abundance of Cap species). In some embodiments, it is desirable to characterize the 3′ UTR of an mRNA, for example to quantify the length of the mRNA polyA tail (also referred to as “Tail”). In some embodiments, it is desirable to map the 5′ UTR of an mRNA to identify whether the mRNA contains Cap, partial Cap, or is uncapped, and the 3′ UTR of an mRNA, for example to quantify the length of the mRNA polyA tail.

The methods of the invention can be used for a variety of purposes where the ability to characterize mRNA is important. For instance, the methods of the invention are useful for monitoring batch-to-batch variability of a synthetic target mRNA or a mRNA sample. The purity of each batch may be determined by determining any differences in the signature profile in comparison to a known signature profile or a theoretical profile of predicted masses from the primary molecular sequence of the mRNA. These signatures are also useful for monitoring the presence of unwanted nucleic acids which may be active components in the sample. The methods may also be performed on at least two samples to determine which sample has better purity or to otherwise compare the purity of the samples.

Thus, in some instances the methods of the invention are used to determine the purity of a RNA sample. The term “pure” as used herein refers to material that has only the target nucleic acid active agents such that the presence of unrelated nucleic acids is reduced or eliminated, i.e., impurities or contaminants, including RNA fragments. For example, a purified RNA sample includes one or more synthetic target or test nucleic acids but is preferably substantially free of other nucleic acids. As used herein, the term “substantially free” is used operationally, in the context of analytical testing of the material. Preferably, purified material substantially free of impurities or contaminants is at least 95% pure; more preferably, at least 98% pure, and more preferably still at least 99% pure. In some embodiments a pure RNA sample is comprised of 100% of the target or test RNAs and includes no other RNA. In some embodiments it only includes a single type of target or test RNA.

Any mRNA may be characterized in accordance with some embodiments of the technology described herein. In some embodiments, methods provided herein comprise characterizing a therapeutic mRNA. In some embodiments, methods provided herein comprise characterizing a target mRNA. In some embodiments, methods provided herein comprise characterizing a target mRNA in a mRNA sample. In some embodiments, methods provided herein comprise characterizing an in vitro transcribed (IVT) mRNA. In some embodiments, a target mRNA is and in vitro transcribed (IVT) mRNA and is considered a synthetic mRNA.

In some embodiments, characterizing a mRNA comprises assigning a signature profile to the mRNA. A “signature profile” of a target mRNA” is a signature generated from an mRNA sample suspected of having a target mRNA based on fragments generated by digestion with a particular RNase enzyme. For example, digestion of an mRNA with RNase T1 and subsequent analysis of the resulting plurality of mRNA fragments by HPLC or mass spec produces a trace or mass profile, or signature that can only be created by digestion of that particular mRNA with RNase T1.

In other embodiments, target mRNA is digested with RNase H. RNase H cleaves the 3′-O-P bond of RNA in a DNA/RNA duplex substrate to produce 3′-hydroxyl and 5′-phosphate terminated products. Therefore, specific nucleic acid (e.g., DNA, RNA, or a combination of DNA and RNA) oligos can be designed to anneal to the target mRNA, and the resulting duplexes digested with RNase H to generate a unique fragment pattern (resulting in a unique mass profile) for a given test mRNA.

Once the signature of a mRNA sample is determined it can be compared with a known signature profile for a target mRNA. A “known signature profile for a target mRNA” as used herein refers to a control signature or fingerprint that uniquely identifies the target mRNA. The known signature profile for a target mRNA may be generated based on digestion of a pure sample and compared to the target signature profile. Alternatively it may be a known control signature, stored in a electronic or non-electronic data medium. For example, a control signature may be a theoretical signature based on predicted masses from the primary molecular sequence of a particular RNA (e.g., a target mRNA).

Various batches of mRNA (e.g., test mRNA) can be digested under the same conditions and compared to the signature of the pure mRNA to identify impurities or contaminants (e.g., additives, such as chemicals carried over from IVT reactions, or incorrectly transcribed mRNA) or to a known signature profile for the target mRNA. The identity of a target mRNA may be confirmed if the signature of the target mRNA shares identity with the known signature profile for a target mRNA. In some embodiments, the signature of the test mRNA shares at least 60%, at least 65%, at least 70%, at least 80%, at least 90%, at least 95%, at least 99%, or at least 99.9% identity with the known mRNA signature.

In some embodiments, various batches of mRNA can be digested under the same conditions in a high throughput fashion. For example, each mRNA sample of a batch may be placed in a separate well or wells of a multi-well plate and digested simultaneously with an RNase. A multi-well plate can comprise an array of 6, 24, 96, 384 or 1536 wells. However, the skilled artisan recognizes that multi-well plates may be constructed into a variety of other acceptable configurations, such as a multi-well plate having a number of wells that is a multiple of 6, 24, 96, 384 or 1536. For example, in some embodiments, the multi-well plate comprises an array of 3072 wells (which is a multiple of 1536). The number of mRNA samples digested simultaneously (e.g., in a multi-well plate) can vary. In some embodiments, at least two mRNA samples are digested simultaneously. In some embodiments, between 2 and 96 mRNA samples are digested simultaneously. In some embodiments, between 2 and 384 mRNA samples are digested simultaneously. In some embodiments, between 2 and 1536 mRNA samples are digested simultaneously. The skilled artisan recognizes that mRNA samples being digested simultaneously can each encode the same protein, or different proteins (e.g., mRNA encoding variants of the same protein, or encoding a completely different protein, such as a control mRNA).

As used herein, the term “digestion” refers to the enzymatic degradation of a biological macromolecule. Biological macromolecules can be proteins, polypeptides, or nucleic acids (e.g., DNA, RNA, mRNA), or any combination of the foregoing. Generally, the enzyme that mediates digestion is a protease or a nuclease, depending upon the substrate on which the enzyme performs its function. Proteases hydrolyze the peptide bonds that link amino acids in a peptide chain. Examples of proteases include but are not limited to serine proteases, threonine proteases, cysteine proteases, aspartase proteases, and metalloproteases. Nucleases cleave phosphodiester bonds between nucleotide subunits of nucleic acids. Generally, nucleases can be classified as deoxyribonucleases, or DNase enzymes (e.g., nucleases that cleave DNA), and ribonucleases, or RNase enzymes (e.g., nucleases that cleave RNA). Examples of DNase enzymes include exodeoxyribonucleases, which cleave the ends of DNA molecules, and restriction enzymes, which cleave specific sequences with a DNA sequence.

The amount of target mRNA that is digested can vary. In some embodiments that amount of target mRNA that is digested ranges from about 1 ng to about 100 μg. In some embodiments, the amount of target mRNA that is digested ranges from about 10 ng to about 80 μg. In some embodiments, the amount of target mRNA that is digested ranges from about 100 ng to about 1000 μg. In some embodiments, the amount of target mRNA that is digested ranges from about 500 ng to about 40 μg. In some embodiments, the amount of target mRNA that is digested ranges from about 1 μg to about 35 μg. In some embodiments, the amount of mRNA that is digested is about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, or about 30 μg.

The disclosure relates, in part, to the discovery that RNase enzymes can be used to digest mRNA to create a unique population of RNA fragments, or a “signature”. Examples of RNase enzymes include but are not limited to RNase A, RNase H, RNase III, RNase L, RNase P, RNase E, RNase PhyM, RNase T1, RNase T2, RNase U2, RNase V, RNase PH, RNase R, RNase D, RNase T, polynucleotide phosphorylase (PNPase), oligoribonuclease, exoribonuclease I, and exoribonuclease II. In some embodiments, RNase T1 or RNase A is used to determine the identity of a test mRNA. In some embodiments, RNase H is used to determine the identity of a test mRNA. In some embodiments, a test mRNA is a synthetic mRNA made by an IVT process.

The concentration of RNase enzyme used in methods described by the disclosure can vary depending upon the amount of mRNA to be digested. However, in some embodiments, the amount of RNase enzyme ranges between about 0.1 Unit and about 500 Units of RNase. In some embodiments, the amount of RNase enzyme ranges from about 0.1 U to about 1 U, 1 U to about 5 U, 2 U to about 200 U, 10 U to about 450 U, about 20 U to about 400 U, about 30 U to about 350 U, about 40 U to about 300 U, about 50 U to about 250 U, or about 100 U to about 200 U.

The skilled artisan also recognizes that RNase enzymes can be derived from a variety of organisms, including but not limited to animals (e.g., mammals, humans, cats, dogs, cows, horses, etc.), bacteria (e.g., E. coli, S. aureus, Clostridium spp., etc.), and mold (e.g., Aspergillus oryzae, Aspergillus niger, Dictyostelium discoideum, etc.). RNase enzymes may also be recombinantly produced. For example, a gene encoding an RNase enzyme from one species (e.g., RNase T1 from A. oryzae) can be heterologously expressed in a bacterial host cell (e.g., E. coli) and purified. In some embodiments, the digestion is performed by an A. oryzae RNase T1 enzyme.

In some embodiments, the digestion is performed in a buffer. As used herein, the term “buffer” refers to a solution that can neutralize either an acid or a base in order to maintain a stable pH. Examples of buffers include but are not limited to Tris buffer (e.g., Tris-Cl buffer, Tris-acetate buffer, Tris-base buffer), urea buffer, bicarbonate buffer (e.g., sodium bicarbonate buffer), HEPES (4-2-hydroxyethyl-1-piperazineethanesulfonic acid) buffer, MOPS (3-(N-morpholino)propanesulfonic acid) buffer, PIPES (piperazine-N,N2-bis(2-ethanesulfonic acid)) buffer, and Triethylammonium acetate (TEAAc buffer). A buffer can also contain more than one buffering agent, for example Tris-Cl and urea. The concentration of each buffering agent in a buffer can range from about 1 mM to about 10 M. In some embodiments, the concentration of each buffering agent in a buffer ranges from about 1 mM to about 20 mM, about 10 mM to about 50 mM, about 25 mM to about 100 mM, about 75 mM to about 200 mM, about 100 mM to about 500 mM, about 250 mM to about 1 M, about 500 mM to about 3 M, about 1 M to about 5 M, about 3 M to about 8 M, or about 5 M to about 10 M.

Generally, the pH maintained by a buffer can range from about pH 6.0 to about pH 10.0. In some embodiments, the pH can range from about pH 6.8 to about 7.5. In some embodiments, the pH is about pH 6.5, about pH 6.6, about pH 6.7, about pH 6.8, about pH 6.9, about pH 7.0, about pH 7.1, about pH 7.2, about pH 7.3, about pH 7.4, about pH 7.5, about pH 7.6, about pH 7.7, about pH 7.8, about pH 7.9, about pH 8.0, about pH 8.1, about pH 8.2, about pH 8.3, about pH 8.4, about pH 8.5, about pH 8.6, about pH 8.7, about pH 8.8, about pH 8.9, about pH 9.0, about pH 9.1, about pH 9.2, about pH 9.3, about pH 9.4, about pH 9.5, about pH 9.6, about pH 9.7, about pH 9.8, about pH 9.9, or about pH 10.

In some embodiments, a buffer further comprises a chelating agent. Examples of chelating agents include, but are not limited to, ethylenediaminetetraacetic acid (EDTA), ethylene glycol tetra acetic acid (EGTA), dimercapto succinic acid (DMSA), and 2,3-dimercapto-1-propanesulfonic acid (DMPS). In some embodiments, the chelating agent is EDTA (ethylenediaminetetraacetic acid). The concentration of EDTA can range from about 1 mM to about 500 mM. In some embodiments, the concentration of EDTA ranges from about 10 mM to about 300 mM. In some embodiments, the concentration of EDTA ranges from about 20 mM to about 250 mM EDTA.

The skilled artisan recognizes that to facilitate digestion, mRNA can be denatured prior to incubation with an RNase enzyme. In some embodiments, mRNA is denatured at a temperature that is at least 50° C., at least 60° C., at least 70° C., at least 80° C., or at least 90° C. Digestion of a target mRNA can be carried out at any temperature at which the RNase enzyme will perform its intended function. The temperature of a target mRNA digestion reaction can range from about 20° C. to about 100° C. In some embodiments, the temperature of a target mRNA digestion reaction ranges from about 30° C. to about 50° C. In some embodiments, a target mRNA is digested by an RNase enzyme at 37° C.

Digestion with RNase enzymes may lead to the formation of cyclic phosphates and other intermediates (e.g., 2′ or 3′-phosphates) that can interfere with downstream processing (e.g., detection of digested test mRNA fragments). Thus, in some embodiments, an mRNA digestion buffer further comprises agents that disrupt or prevent the formation of intermediates. In some embodiments, the buffer further comprises 2′,3′-Cyclic-nucleotide 3′-phosphodiesterase (CNP) and/or Alkaline Phosphatase, such as Calf Intestinal Alkaline Phosphatase (CIP), or Shrimp Alkaline Phosphatase (SAP). The concentration of each agent that disrupts or prevents formation of intermediates can range from about 10 ng/μL to about 100 ng/μL. In some embodiments, the concentration of each agent ranges from about 15 ng/μL to about 25 ng/μL. Alternatively, or in combination with the above-stated concentration range, the amount of agent can range from about 1 U to about 50 U, about 2 U to about 40 U, about 3 U to about 35 U, about 4 U to about 30 U, about 5 U to about 25 U, or about 10 U to about 20 U. In some embodiments, digestion with RNase enzymes is performed in a digestion buffer not containing CIP and/or CNP.

In some embodiments, a buffer further comprises magnesium chloride (MgCl₂). Generally, MgCl₂ can act as a cofactor for enzyme (e.g., RNase) activity. The concentration of MgCl₂ in the buffer ranges from about 0.5 mM to about 200 mM. In some embodiments, the concentration of MgCl₂ in the buffer ranges from about 0.5 mM to about 10 mM, 1 mM to about 20 mM, 5 mM to about 20 mM, 10 mM to about 75 mM, or about 50 mM to about 150 mM. In some embodiments, the concentration of MgCl₂ in the buffer is about 1 mM, about 5 mM, about 10 mM, about 50 mM, about 75 mM, about 100 mM, about 125 mM, or about 150 mM.

In some embodiments, digestion of a test mRNA comprises two incubation steps: (a) RNase digestion of test mRNA, and (b) processing of digested test mRNA. In some embodiments, digestion of a test mRNA further comprises the step of denaturing test mRNA prior to digestion. The incubation time for each of the above steps (a), (b), and (c) can range from about 1 minute to about 24 hours. In some embodiments, incubation time ranges from about 1 minute to about 10 minutes. In some embodiments, incubation time ranges from about 5 minutes to about 15 minutes. In some embodiments, incubation time ranges from about 30 minutes to about 4 hours (240 minutes). In some embodiments, incubation time ranges from about 1 hour to about 5 hours. In some embodiments, incubation time ranges from about 2 hours to about 12 hours. In some embodiments, incubation time ranges from about 6 hours to about 24 hours.

The skilled artisan recognizes that digestions may be carried out under various environmental conditions based upon the components present in the digestion reaction. Any suitable combination of the foregoing components and parameters may be used. For example, digestion of a test mRNA may be carried out according to the protocol set forth in Table 1.

In some aspects, the disclosure provides a “one-pot” RNase H digestion assay for characterization of nucleic acids (e.g., a target mRNA). Generally, RNase H digestion assays comprise separate steps for (i) annealing a guide strand to a target mRNA and (ii) digesting the guide strand-mRNA duplex. The disclosure relates, in part, to the discovery that guide strand annealing and RNase H digestion steps can be combined into a single step when appropriate conditions (e.g., as set forth in Table 1) are provided. Without wishing to be bound by any particular theory, a one-pot RNase H digestion assay as described by the disclosure, in some embodiments, has a reduced run time and provides higher quality samples for analytical methods (e.g., HPLC/MS, etc.) than methods requiring multiple steps (e.g., separate annealing and digestion steps, etc.).

A “fragment” of a polynucleotide of interest comprises a series of consecutive nucleotides from the sequence of said test RNA. By way of example, a “fragment” of a polynucleotide of interest may comprise (or consist of) at least 1 at least 2, at least 5, at least 10, at least 20, at least 30 consecutive nucleotides from the sequence of the polynucleotide (e.g., at least 1 at least 2, at least 5, at least 10, at least 20, at least 30, at least 35, 50, 75, 100, 150, 200, 250, 300, 350, 400, 450, 500, 550, 600, 650, 700, 750, 800 850, 900, 950 or 1000 consecutive nucleic acid residues of said polynucleotide). A fragment of a polynucleotide (e.g., an mRNA fragment) can consist of the same nucleotide sequence as another fragment, or consist of a unique nucleotide sequence.

A “plurality of mRNA fragments” refers to a population of at least two mRNA fragments. mRNA fragments comprising the plurality can be identical, unique, or a combination of identical and unique (e.g., some fragments are the same and some are unique). The skilled artisan recognizes that fragments can also have the same length but comprise different nucleotide sequences (e.g., CACGU, and AAAGC are both five nucleotides in length but comprise different sequences). In some embodiments, a plurality of mRNA fragments is generated from the digestion of a single species of mRNA. A plurality of mRNA fragments can be at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 400, or at least 500 mRNA fragments. In some embodiments, a plurality of mRNA fragments comprises more than 500 mRNA fragments.

The plurality of fragments is physically separated. As used herein, the term “physically separated” refers to the isolation of mRNA fragments based upon a selection criterion. For example, a plurality of mRNA fragments resulting from the digestion of a test mRNA can be physically separated by chromatography or mass spectrometry. In some embodiments, fragments of a test mRNA can be physically separated by capillary electrophoresis to generate an electropherogram. Examples of chromatography methods include size exclusion chromatography and high-performance liquid chromatography (HPLC). Examples of mass spectrometry physical separation techniques include electrospray ionization mass spectrometry (ESI-MS) and matrix-assisted laser desorption ionization time of flight (MALDI-TOF). In some embodiments, each of fragment of the plurality of mRNA fragments is detected during the physical separation. For example, a UV spectrophotometer coupled to a HPLC machine can be used to detect the mRNA fragments during physical separation (e.g., an absorbance spectrum profile). The resulting data, also called a “trace” provides a graphical representation of the composition of the plurality of mRNA fragments. In another embodiment, a mass spectrophotometer generates mass data during the physical separation of a plurality of mRNA fragments. The graphic depiction of the mass data can provide a “mass fingerprint” that identifies the contents of the plurality of mRNA fragments.

Mass spectrometry encompasses a broad range of techniques for identifying and characterizing compounds in mixtures. Different types of mass spectrometry-based approaches may be used to analyze a sample to determine its composition. Mass spectrometry analysis involves converting a sample being analyzed into multiple ions by an ionization process. Each of the resulting ions, when placed in a force field, moves in the field along a trajectory such that its acceleration is inversely proportional to its mass-to-charge ratio. A mass spectrum of a molecule is thus produced that displays a plot of relative abundances of precursor ions versus their mass-to-charge ratios. When a subsequent stage of mass spectrometry, such as tandem mass spectrometry, is used to further analyze the sample by subjecting precursor ions to higher energy, each precursor ion may undergo disassociation into fragments referred to as product ions. Resulting fragments can be used to provide information concerning the nature and the structure of their precursor molecule.

MALDI-TOF (matrix-assisted laser desorption ionization time of flight) mass spectrometry provides for the spectrometric determination of the mass of poorly ionizing or easily-fragmented analytes of low volatility by embedding them in a matrix of light-absorbing material and measuring the weight of the molecule as it is ionized and caused to fly by volatilization. Combinations of electric and magnetic fields are applied on the sample to cause the ionized material to move depending on the individual mass and charge of the molecule. U.S. Pat. No. 6,043,031, issued to Koster et al., describes an exemplary method for identifying single-base mutations within DNA using MALDI-TOF and other methods of mass spectrometry.

HPLC (high performance liquid chromatography) is used for the analytical separation of bio-polymers, based on properties of the bio-polymers. HPLC can be used to separate nucleic acid sequences based on size and/or charge. A nucleic acid sequence having one base pair difference from another nucleic acid can be separated using HPLC. Thus, nucleic acid samples, which are identical except for a single nucleotide may be differentially separated using HPLC, to identify the presence or absence of a particular nucleic acid fragments. Preferably the HPLC is HPLC-UV.

The data generated using the methods of the invention can be processed individually or by a computer. For instance, a computer-implemented method for generating a data structure, tangibly embodied in a computer-readable medium, representing a data set representative of a signature profile of an RNA sample may be performed according to the invention.

Some embodiments relate to at least one non-transitory computer-readable storage medium storing computer-executable instructions that, when executed by at least one processor, perform a method of identifying an RNA in a sample.

Thus, some embodiments provide techniques for processing MS/MS data that may identify impurities in a sample with improved accuracy, sensitivity and speed. The techniques may involve structural identification of an RNA fragment regardless of whether it has been previously identified and included in a reference database. A scoring approach may be utilized that allows determining a likelihood of an impurity being present in a sample, with scores being computed so that they do not depend on techniques used to acquire the analyzed mass spectrometry data.

In some embodiments the known signature profile for known mRNA data may be computationally generated, or computed, and stored, for example, in a first database. The first database may store any type of information on the RNA, including an identifier of each RNA fragment to form a complete signature and any other suitable information. In some embodiments, a score may be computed for each set of computed fragments retrieved from a second database including the known signatures, the score indicating correlation between the set of known signatures and the set of experimentally obtained fragments. To compute the score, for example, each fragment in a set of computed fragments matching a corresponding fragment in the set of experimentally obtained fragments may be assigned a weight based on a relative abundance of the experimentally obtained fragment. A score may thus be computed for each set of computed fragments based on weights assigned to fragments in that set. The scores may then be used to identify difference between the RNA sample and the known sequence.

A computer system that may implement the above as a computer program typically may include a main unit connected to both an output device which displays information to a user and an input device which receives input from a user. The main unit generally includes a processor connected to a memory system via an interconnection mechanism. The input device and output device also may be connected to the processor and memory system via the interconnection mechanism.

An illustrative implementation of a computer system that may be used in connection with some embodiments may be used to implement any of the functionality described above. The computer system may include one or more processors and one or more computer-readable storage media (i.e., tangible, non-transitory computer-readable media), e.g., volatile storage and one or more non-volatile storage media, which may be formed of any suitable data storage media. The processor may control writing data to and reading data from the volatile storage and the non-volatile storage device in any suitable manner, as embodiments are not limited in this respect. To perform any of the functionality described herein, the processor may execute one or more instructions stored in one or more computer-readable storage media (e.g., volatile storage and/or non-volatile storage), which may serve as tangible, non-transitory computer-readable media storing instructions for execution by the processor.

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation comprises at least one computer-readable storage medium (i.e., at least one tangible, non-transitory computer-readable medium), such as a computer memory (e.g., hard drive, flash memory, processor working memory, etc.), a floppy disk, an optical disk, a magnetic tape, or other tangible, non-transitory computer-readable medium, encoded with a computer program (i.e., a plurality of instructions), which, when executed on one or more processors, performs above-discussed functions. The computer-readable storage medium can be transportable such that the program stored thereon can be loaded onto any computer resource to implement techniques discussed herein. In addition, it should be appreciated that the reference to a computer program which, when executed, performs above-discussed functions, is not limited to an application program running on a host computer. Rather, the term “computer program” is used herein in a generic sense to reference any type of computer code (e.g., software or microcode) that can be employed to program one or more processors to implement above-techniques.

Further aspects related to characterizing a target mRNA or RNA sample are provided in U.S. patent application publication number US 2018/0274009, entitled “METHODS AND COMPOSITIONS FOR RNA MAPPING,” filed Jun. 6, 2018, the entire contents of which are incorporated herein by reference.

### Methods of Selecting an Isolated Nucleic Acid

Aspects of the present disclosure relate to methods of selecting an isolated nucleic acid described herein for analyzing and characterizing a RNA sample (e.g., a target mRNA).

In some embodiments, methods of selecting an isolated nucleic acid comprise digesting a mRNA hybridized to an isolated nucleic acid provided herein with an RNase enzyme to produce a plurality of mRNA fragments; physically separating the plurality of mRNA fragments; generating a signature profile of the mRNA by detecting the plurality of mRNA fragments; comparing the signature profile with a known mRNA signature profile, and selecting the isolated nucleic acid based on the comparison of the signature profile with the known RNA signature profile.

An isolated nucleic acid may be selected based on any aspect of a signature profile, e.g., a signature profile of a target mRNA. In some embodiments, the signature profile is in the form of an absorbance spectrum or a mass spectrum. In some embodiments, the signature profile comprises determining Cap structure of the mRNA based upon comparison of the signature profile with the known RNA signature profile. In some embodiments, the signature profile comprises a raw mass spectrometry profile. In some embodiments, the signature profile comprises a retention time.

In some embodiments, selecting an isolated nucleic acid comprises comparing a signature profile of a target mRNA with a known mRNA signature profile. In some embodiments, selecting an isolated nucleic acid comprises selecting an isolated nucleic acid described herein. In some embodiments, selecting an isolated nucleic acid comprises selecting an isolated nucleic acid from the group consisting of SEQ ID NOs: 3-15. In some embodiments, selecting an isolated nucleic acid comprises selecting at least one of SEQ ID NOs: 3-15. In some embodiments, selecting an isolated nucleic acid comprises selecting at least two of SEQ ID NOs: 3-15. In some embodiments, selecting an isolated nucleic acid comprises selecting at least three of SEQ ID NOs: 3-15.

### EXAMPLES

In order that the invention described herein may be more fully understood, the following examples are set forth. The examples described in this application are offered to illustrate the systems and methods provided herein and are not to be construed in any way as limiting their scope.

### Example 1

### Materials and Methods

### RNase H Guides

Cap:
(SEQ ID NO: 16)
5′-mC*mU*mU*mA*mC*mU*mC*mU*mU*mC*mU*mU*mU*mU*mC*
dTdCdTdCmU*mU*mA-3′
(mN* = 2′OMe Phosphorothioate)
Tail:
(SEQ ID NO: 17)
5′-mCmAmGmAmCdTdTdTdAmUmUmCmAmA
(mN = 2′OMe)

### RNase H Digestion Mixtures

The following components were mixed to prepare RNase H digestion mixtures:

| | | | 1x Cap | 20x Cap |
|---|---|---|---|---|
| | 1x Cap | 1x Tail | & Tail | & Tail |
| Component | (μL) | (μL) | (μL) | (μL) |
| RNase H Cap Guide | 0.1 | — | 0.1 | 2 |
| RNase H Tail Guide | — | 0.9 | 0.9 | 18 |
| LCMS Water | 0.9 | 0.1 | — | — |
| Hybridase (2 U/μL) | 2 | 2 | 2 | 40 |
| Calf Intestinal | 2 | 2 | 2 | 40 |
| Phosphatase (CIP) | | | | |
| (2 U/μL) | | | | |
| 10X RNase H | 3 | 3 | 3 | 60 |
| digestion buffer | | | | |
| Total Volume | 8 | 8 | 8 | 160 |

### Sample Preparation and Analysis

The following reactants were mixed in PCR plates:

| Example | Volume |
|---|---|
| mRNA test sample at 0.8-1 mg/mL | 20 μL |
| RNase H Digestion Mixture | 8 μL |
| Total Volume per sample | 28 μL |

Then plates were sealed, vortexed gently to mix, and centrifuged at 1000 rpm for 30 seconds. Plates were incubated in a thermocycler for 15 minutes at 65° C., followed by a hold at 5° C. Reactions were stopped by the addition of 5 μL of digestion quench buffer (1 M triethylammonium acetate (TEAA), 250 mM EDTA) to each sample. Samples were then analyzed by LC-MS or HPLC-UV.

### Example 2

### Cap Guide Selection

This example describes the digestion of mRNA cap region by RNase H. Breifly, RNase H guide strands specific for Cap regions were used to digest a mRNA. LC-MS analysis was then performed, and the following data were analyzed: (i) Cap identification and relative quantification; (ii) polyA tail length identification and relative quantification; optionally, (iii) total digest and mapping.

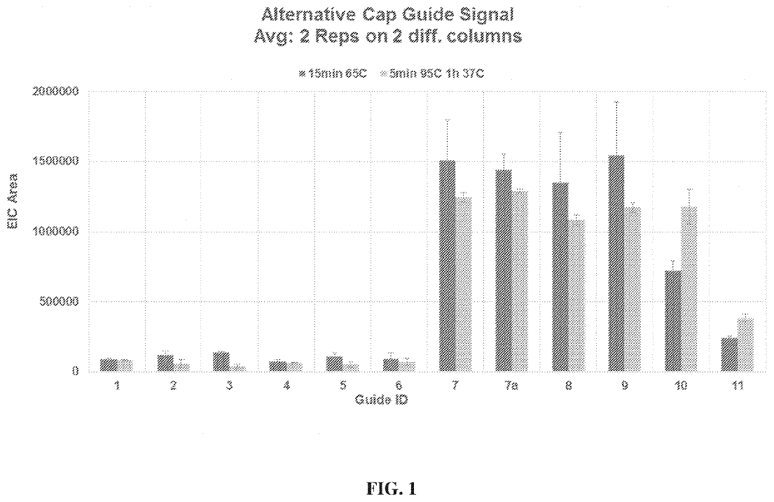

FIG. 1 shows representative extracted ion chromatogram (EIC) data for mRNA digested with various cap variants described herein. Increased levels of RNase H Cap signal was detected for Cap variants described as Guide ID NO: 7, 7a, 8, and 9.

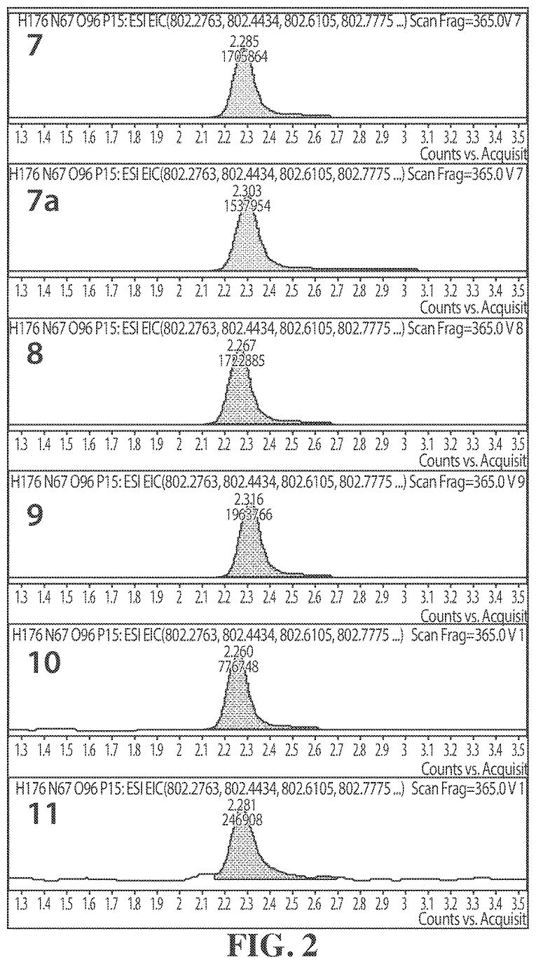

The ability to direct RNase H specificity and flexibility in the length of the RNase H guide strand significantly advances one's ability to direct the retention times of the RNase H target fragment (e.g., cap fragment) and the RNase H guide itself, allowing one to prevent undesired co-elution, and consequently, yield relatively consistent reliable and clean LC-MS data. It should be noted that it is expected that in some cases, RNase H cleavage of mRNA may not total, but succeed in most cases where DNAzyme fails. Therefore, RNase H substrate specificity was examined Cleavage efficiency of RNase H relative to RNA bases 5′ and 3′ of the cut site was evaluated. Data indicate that no RNase H cleavage occurred 3′ and 5′ of the cut site (FIG. 2).

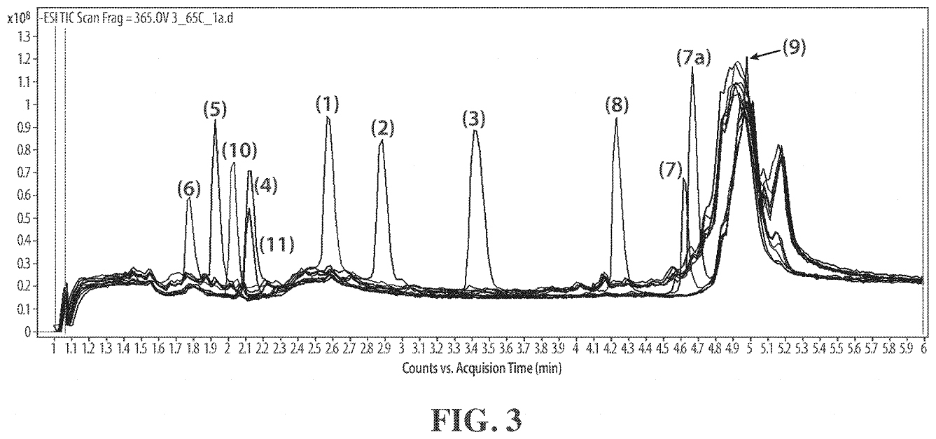

FIG. 3 shows representative raw data for a total ion chromatogram (TIC) of a one-pot cap RNase H assay. No overlap with Cap fragments were observed. Retention times were more compatible for cap variants described as Guide ID NO: 7, 7a, and 8 as compared to other cap variants.

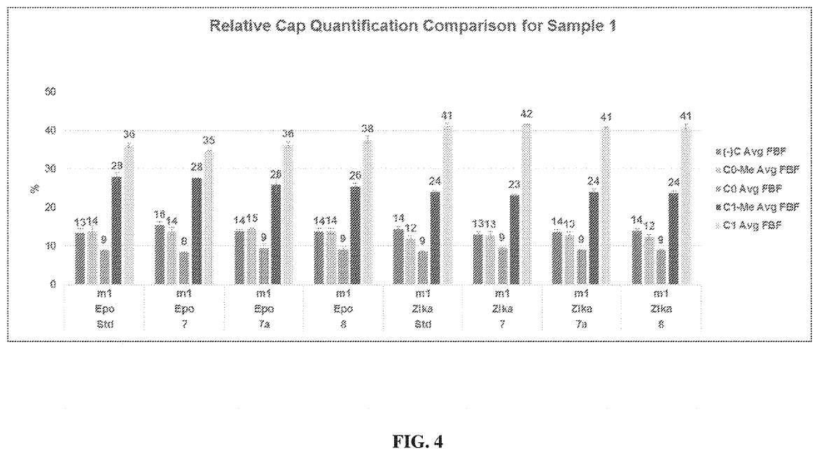

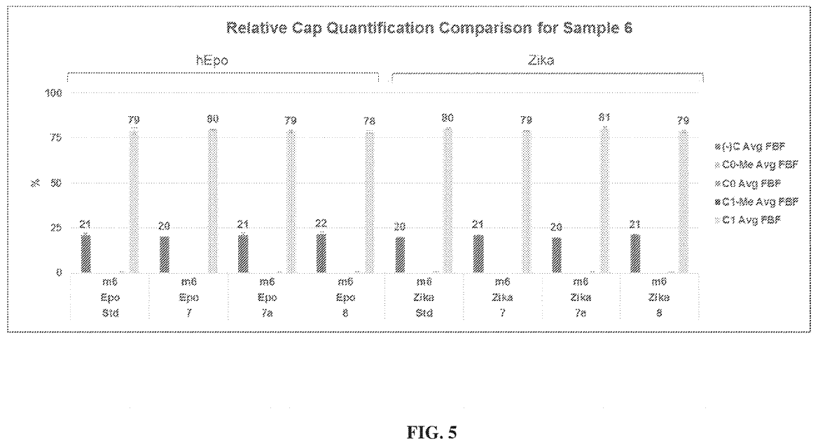

RNase H guide strands specific for Cap regions were used to digest an mRNA encoding human EPO (hEPO) and a viral antigen (viral Ag). LC-MS analysis was then performed and then cap identification and relative quantification was performed for Sample 1 (FIG. 4) and Sample 6 (FIG. 5).

### Example 3

### Modified Cap Guides

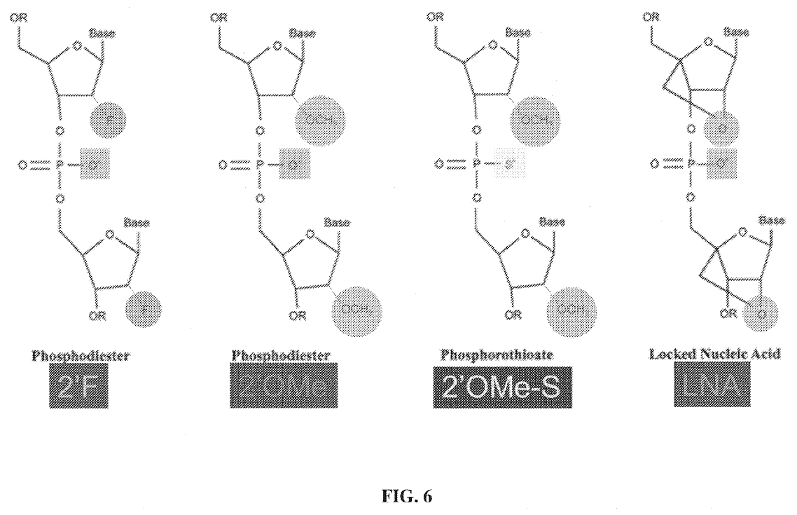

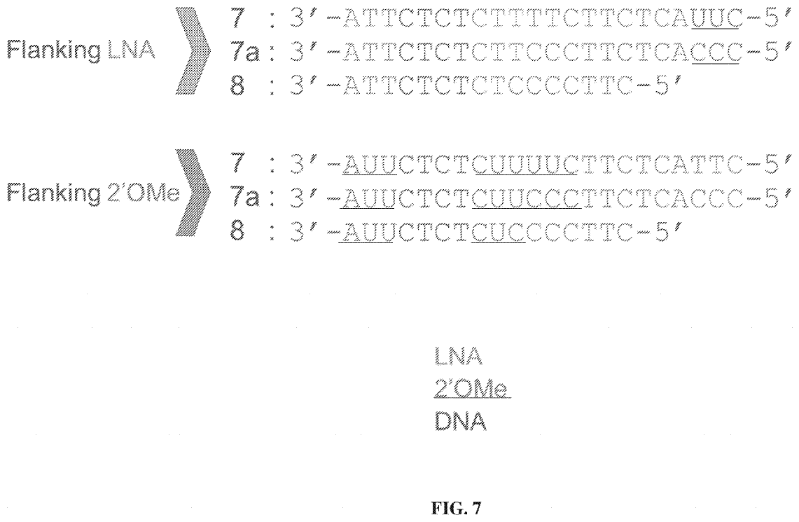

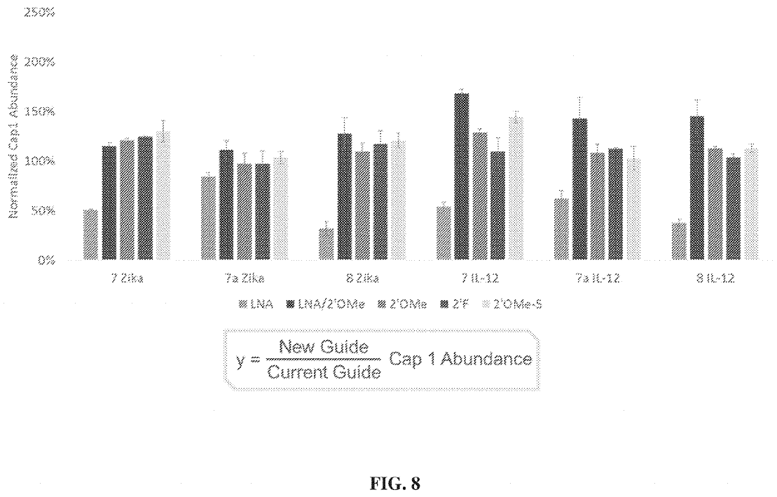

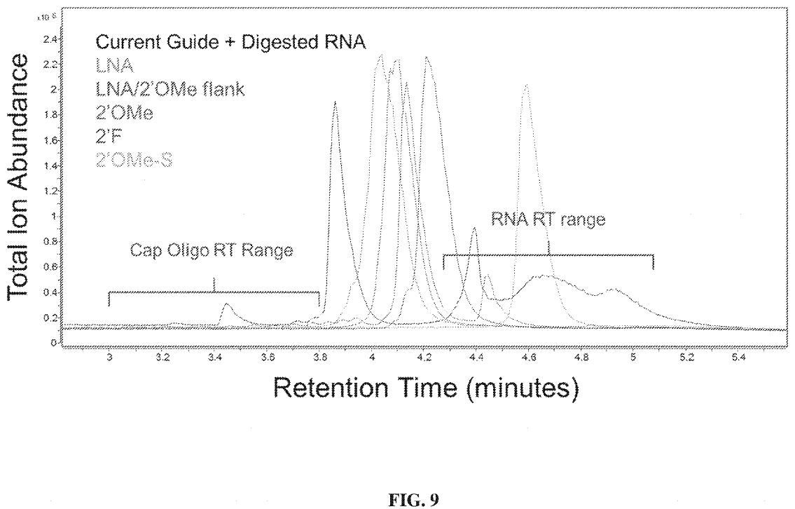

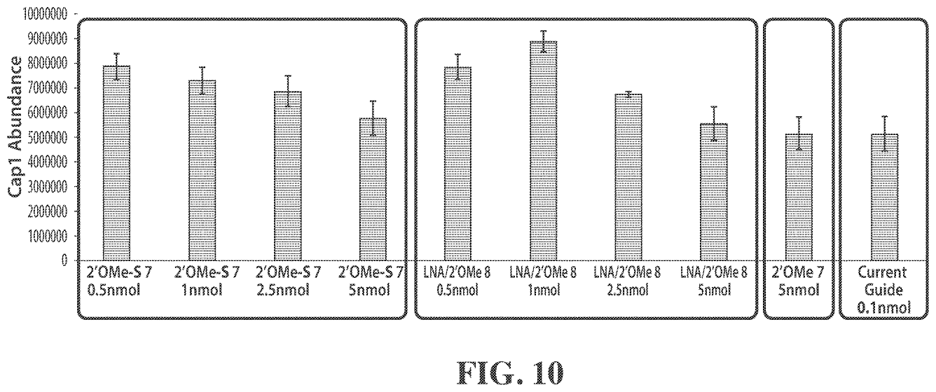

Modified Cap guides (modified versions of Guide ID NO: 7, 7a, 8, and 9) were tested in one-pot cap RNase H assays. Representative structures of backbone modifications of interest are shown in FIG. 6. Representative Cap guide sequences comprising flanking LNA or flanking LNA/2′OMe sequences are shown in FIG. 7. Modified cap guides were used to digest an mRNA encoding a viral Ag or IL-12. Representative data of normalized Capl abundance is shown in FIG. 8. Representative total ion chromatogram (TIC) data for retention time is shown in FIG. 9. mRNA encoding a viral Ag or IL-12 was digested with increasing concentrations of modified cap variants. Representative data of Cap1 abundance is shown in FIG. 10.

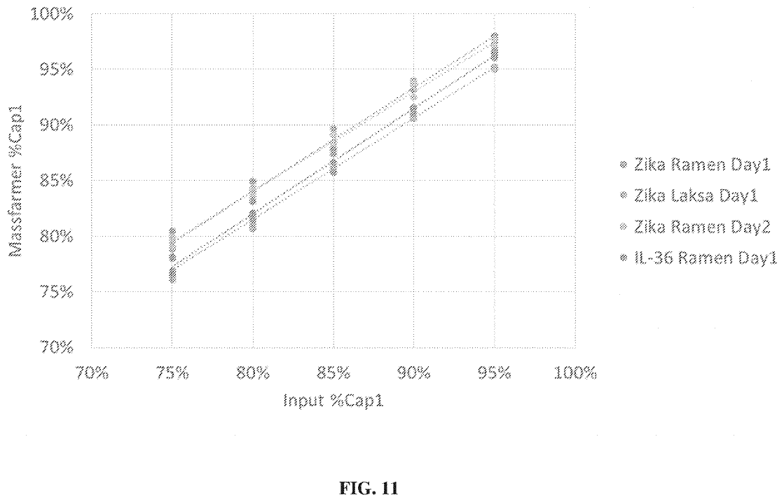

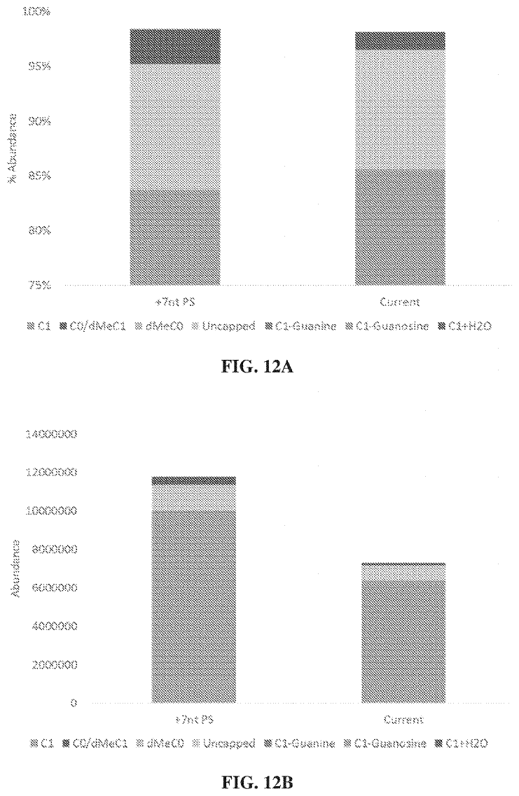

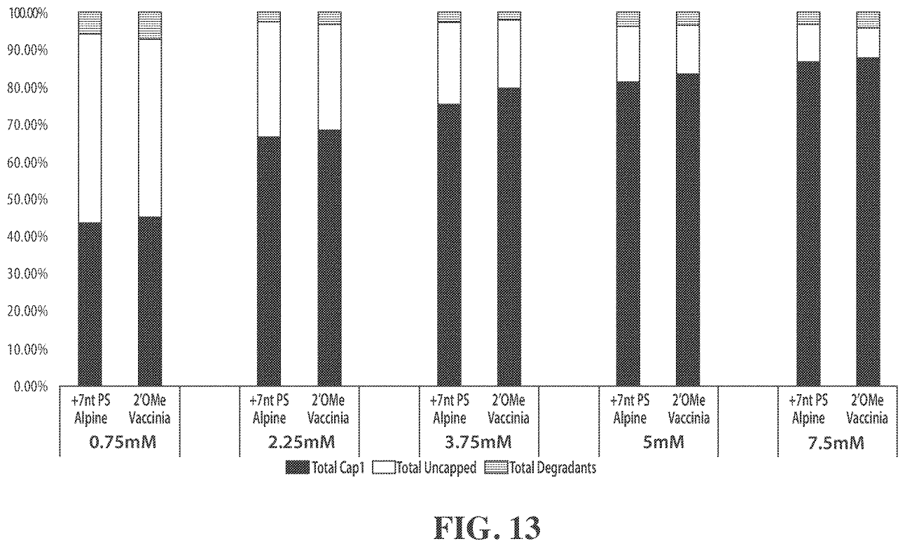

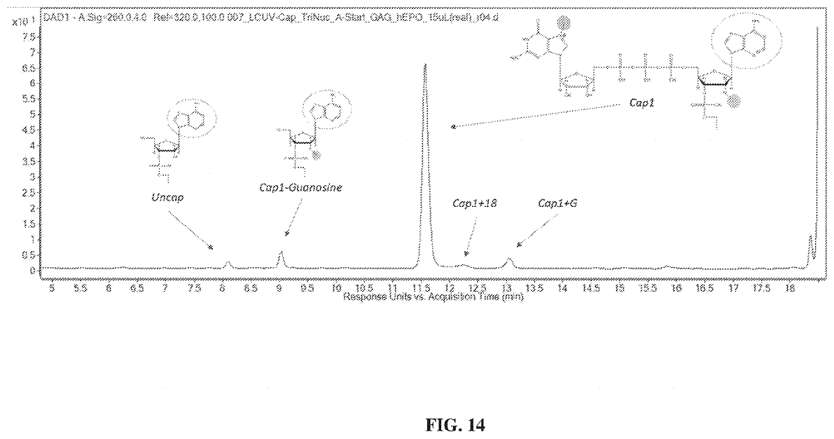

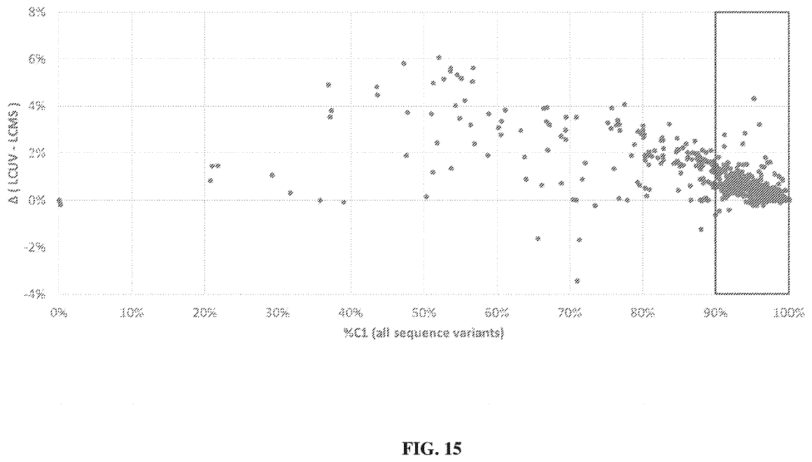

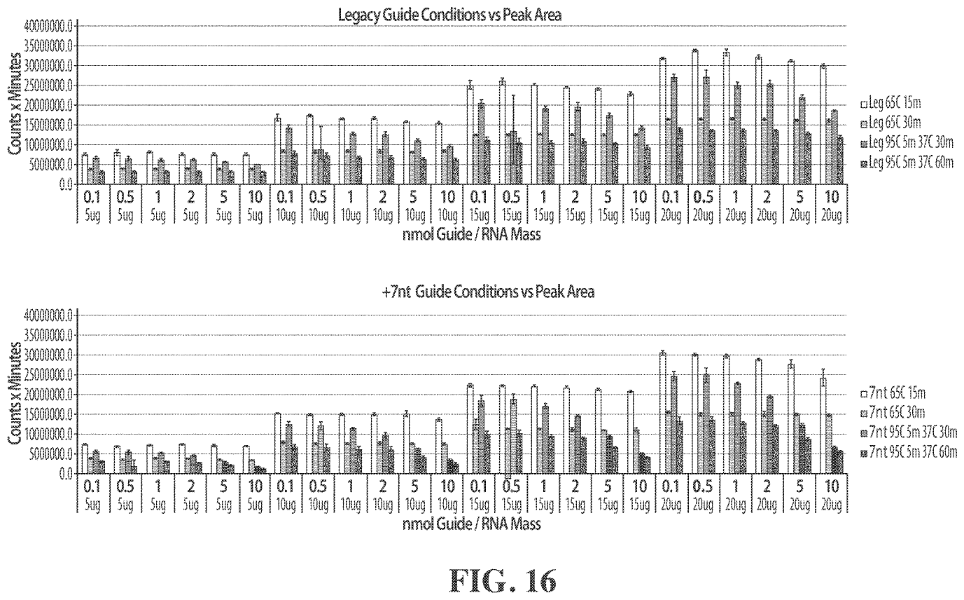

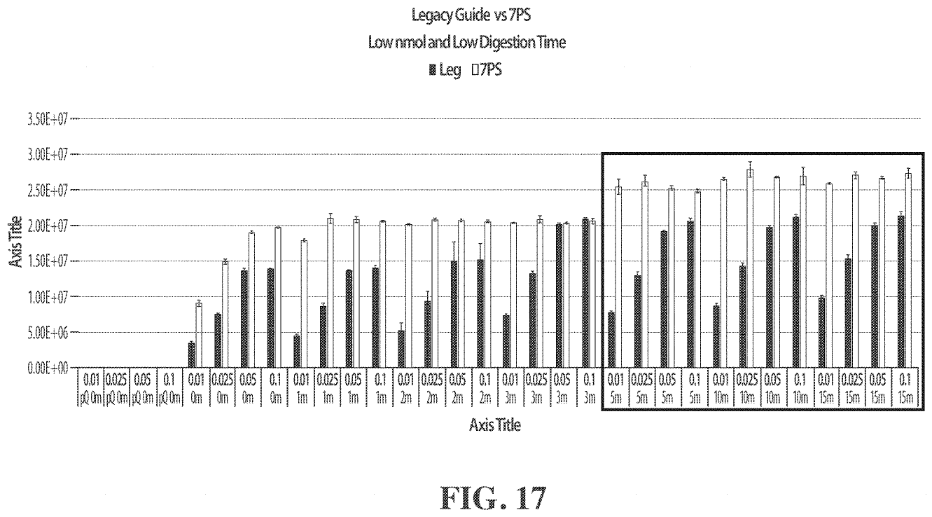

The relationship between % Cap1 and Input % Cap1 was linear for Guide ID NO: 7 containing the 2′OMe Phosphorothioate (7PS) as shown in FIG. 11. 7PS provided similar results with respect to percent abundance (FIG. 12A) and raw abundance (FIG. 12B) compared to a control cap guide (current). Slightly more uncapped mRNA was detected in samples comprising 7PS compared to the control cap (FIG. 13). Representative LC-UV data is shown in FIG. 14, and effects of sequence variants is shown in FIG. 15. Representative data showing results of Cap guide 7PS and control at various conditions is shown in FIGS. 16-17.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. An isolated nucleic acid represented by the formula from 5′ to 3′:
[R]_{q}D₁D₂D₃D₄[R]ₚ
wherein each R is an unmodified or modified RNA nucleotide, D is a deoxyribonucleotide base and each of q and p are independently an integer between 0 and 50, wherein the isolated nucleic acid hybridizes to an mRNA at a position that is at least 7 nucleotides downstream of the first nucleotide of the mRNA, and
wherein hybridization of the isolated nucleic acid to the mRNA in the presence of RNase H results in cleavage of the mRNA by the RNase H.

2. The isolated nucleic acid of claim 1, wherein the mRNA comprises a 5′ UTR set forth in SEQ ID NO: 1 or SEQ ID NO: 2.

3. The isolated nucleic acid of claim 1 or 2, wherein at least one R comprises:
i) a modified RNA nucleotide, optionally a 2′-O-methyl modified RNA base, a 2′ Fluoro modified RNA base, a peptide nucleic acid (PNA), or locked nucleic acid (LNA);
ii) a modified backbone, optionally wherein the modified backbone is a phosphorothioate backbone; or
iii) a combination of i) and ii).

4. The isolated nucleic acid of any one of claims 1 to 3, wherein D₁ and D₃ comprise cytosine (C), and D₂ and D₄ comprise thymine (T).

5. An isolated nucleic acid represented by the formula from 5′ to 3′:
[R] _{q}D₁D₂D₃D₄[R]ₚ
wherein each R is an unmodified or modified RNA base, D is a deoxyribonucleotide base and each of q and p are independently an integer between 0 and 50,
wherein D₁ and D₃ comprise cytosine (C), and D₂ and D₄ comprise thymine (T), and
wherein hybridization of the isolated nucleic acid to a mRNA 5′ untranslated region (5′ UTR) in the presence of RNase H results in cleavage of the mRNA 5′ UTR by the RNase H.

6. The isolated nucleic acid of any one of claims 1 to 5, wherein at least one of D₁, D₂ D₃, and D₄ are unmodified deoxyribonucleotide bases.

7. The isolated nucleic acid of any one of claim 1 or 6, wherein at least one of D₁, D₂ D₃, and D₄ are modified deoxyribonucleotide bases.

8. The isolated nucleic acid of claim 7, wherein the modified deoxyribonucleotide base is 5-nitroindole, Inosine, 4-nitroindole, 6-nitroindole, 3-nitropyrrole, a 2-6-diaminopurine, 2-amino-adenine, or 2-thio-thiamine.

9. The isolated nucleic acid of any one of claims 1 to 8, wherein the cleavage of the mRNA 5′ UTR by the RNase H results in liberation of an intact mRNA Cap.

10. The isolated nucleic acid of any one of claims 1 to 9, wherein the mRNA is in vitro transcribed (IVT) RNA.

11. The isolated nucleic acid of any one of claims 1 to 10, wherein the isolated nucleic acid is selected from the sequences set forth in Table 2.

12. The isolated nucleic acid of claim 11, wherein the isolated nucleic acid is SEQ ID NO: 3 or SEQ ID NO: 4.

13. The isolated nucleic acid of claim 11, wherein the isolated nucleic acid is SEQ ID NO: 5 or SEQ ID NO: 6.

14. The isolated nucleic acid of claim 11, wherein the isolated nucleic acid is SEQ ID NO: 7 or SEQ ID NO: 8.

15. The isolated nucleic acid of claim 11, wherein the isolated nucleic acid is SEQ ID NO: 9 or SEQ ID NO: 10.

16. The isolated nucleic acid of claim 11, wherein the isolated nucleic acid is SEQ ID NO: 11 or SEQ ID NO: 12.

17. The isolated nucleic acid of claim 11, wherein the isolated nucleic acid is SEQ ID NO: 13 or SEQ ID NO: 14.

18. The isolated nucleic acid of claim 11, wherein the isolated nucleic acid is SEQ ID NO: 15.

19. A composition comprising a plurality of isolated nucleic acids, wherein each of the isolated nucleic acids individually is an isolated nucleic acid as described in any one of claims 1 to 18.

20. The composition of claim 19, wherein the plurality is three or more isolated nucleic acids.

21. The composition of claim 19 or 20 further comprising a buffer, and optionally, RNase H enzyme.

22. A method of selecting an isolated nucleic acid, the method comprising:
digesting a mRNA hybridized to an isolated nucleic acid as described in any one of claims 1 to 18 with an RNase enzyme to produce a plurality of mRNA fragments;
physically separating the plurality of mRNA fragments;
generating a signature profile of the mRNA by detecting the plurality of mRNA fragments;
comparing the signature profile with a known mRNA signature profile, and
selecting the isolated nucleic acid based on the comparison of the signature profile with the known RNA signature profile.

23. The method of claim 22, wherein the selecting and/or the detecting comprises a method selected from the group consisting of gel electrophoresis, capillary electrophoresis, high pressure liquid chromatography (HPLC), and mass spectrometry.

24. The method of claim 23, wherein the HPLC is HPLC-UV.

25. The method of claim 23, wherein the mass spectrometry is Electrospray Ionization mass spectrometry (ESI-MS) or Matrix-assisted Laser Desorption/Ionization-Time of Flight (MALDI-TOF) mass spectrometry.

26. The method of any one of claims 22 to 25, wherein the mRNA is mixed with a buffer comprising at least one component selected from the group consisting of urea, EDTA, magnesium chloride (MgCl₂) and Tris prior to digestion.

27. The method of claim 26, wherein the mRNA and the buffer are incubated at a temperature between 60° C. to 100° C.

28. The method of any one of claims 22 to 27 further comprising incubating the mRNA sample with 2′,3′-Cyclic-nucleotide 3′-phosphodiesterase (CNP) following the digestion to produce a CNP treated mRNA sample.

29. The method of claim 28, wherein the incubating of the mRNA with CNP is performed for about 1 hour.

30. The method of claim 28, further comprising incubating the CNP treated mRNA with Calf Intestinal Alkaline Phosphatase (CIP).

31. The method of any one of claims 28 to 30, further comprising incubating the mRNA with an enzymatic inhibitor.

32. The method of claim 31, wherein the enzymatic inhibitor is EDTA.

33. The method of any one of claims 22 to 32, wherein the signature profile is in the form of an absorbance spectrum or a mass spectrum.

34. The method of any one of claims 22 to 33, wherein the isolated nucleic acid is an isolated nucleic acid of any one of claims 1 to 18.

35. The method of any one of claims 22 to 34, wherein the mRNA 5′ untranslated region (5′ UTR) comprises SEQ ID NO: 1 or SEQ ID NO: 2.

36. The method of any one of claims 22 to 35, wherein comparing the signature profile comprises determining Cap structure of the mRNA based upon comparison of the signature profile with the known RNA signature profile.

37. A method for quality control of an RNA pharmaceutical composition, comprising
digesting the RNA pharmaceutical composition with an RNase H enzyme to produce a plurality of RNA fragments;
physically separating the plurality of RNA fragments;
generating a signature profile of the RNA pharmaceutical composition by detecting the plurality of fragments;
comparing the signature profile with a known RNA signature profile; and
determining the quality of the RNA based on the comparison of the signature profile with the known RNA signature profile;
wherein the digesting step comprises contacting the RNA pharmaceutical composition with an isolated nucleic acid of any one of claims 1 to 18, or a pharmaceutical composition of any one of claims 19 to 21 prior to contacting the RNA pharmaceutical composition with an RNase H enzyme.

38. The method of claim 37, wherein the digestion step is performed in the presence of a blocking oligonucleotide.

39. The method of claim 38, wherein the blocking oligonucleotide comprises at least one modified nucleotide, optionally wherein the modification is selected from locked nucleic acid nucleotide (LNA), 2′ OMe-modified nucleotide, and peptide nucleic acid (PNA) nucleotide.

40. The method of claim 38 or 39, wherein the blocking oligonucleotide targets the 5′ untranslated region (5′UTR) or the 3′ untranslated region (3′UTR) of the test mRNA.

41. The method of any one of claims 37 to 40, wherein the mRNA is prepared by in vitro transcription (IVT).

42. The method of any one of claims 37 to 41, wherein the RNA is a therapeutic mRNA.


SEQUENCE LISTING (23 sequences)

SEQ ID NO: 1 - DNA, 47 bp, Artificial Sequence
  Synthetic
gggaaataagagagaaaagaagagtaagaagaaatataagagccacc

SEQ ID NO: 2 - DNA, 57 bp, Artificial Sequence
  Synthetic
gggaaataagagagaaaagaagagtaagaagaaatataagaccccggcgccgccacc

SEQ ID NO: 3 - DNA, 16 bp, Artificial Sequence
  Synthetic
cccuuuauuctctuac

SEQ ID NO: 4 - DNA, 12 bp, Artificial Sequence
  Synthetic
auuctctcuuuu

SEQ ID NO: 5 - DNA, 13 bp, Artificial Sequence
  Synthetic
auuctctcuuuuc

SEQ ID NO: 6 - DNA, 14 bp, Artificial Sequence
  Synthetic
auuctctcuuuucu

SEQ ID NO: 7 - DNA, 11 bp, Artificial Sequence
  Synthetic
uuctctcuuuu

SEQ ID NO: 8 - DNA, 10 bp, Artificial Sequence
  Synthetic
uctctcuuuu

SEQ ID NO: 9 - DNA, 9 bp, Artificial Sequence
  Synthetic
ctctcuuuu

SEQ ID NO: 10 - DNA, 22 bp, Artificial Sequence
  Synthetic
auuctctcuuuucuucucauuc

SEQ ID NO: 11 - DNA, 22 bp, Artificial Sequence
  Synthetic
auuctctcuucccuucucaccc

SEQ ID NO: 12 - DNA, 16 bp, Artificial Sequence
  Synthetic
auuctctcuccccuuc

SEQ ID NO: 13 - DNA, 34 bp, Artificial Sequence
  Synthetic
auuctctcuuuucuucucauucuucuuuauauuc

SEQ ID NO: 14 - DNA, 10 bp, Artificial Sequence
  Synthetic
auuctctcuu

SEQ ID NO: 15 - DNA, 10 bp, Artificial Sequence
  Synthetic
auuctctuac

SEQ ID NO: 16 - DNA, 22 bp, Artificial Sequence
  Synthetic
  modified_base (1)..(15): modified with 2′-O-methyl
  modified_base (1)..(15): modified with phosphorothioate
  misc_feature (16)..(16): deoxythymine
  misc_feature (17)..(17): deoxycytidine
  misc_feature (18)..(18): deoxythymine
  misc_feature (19)..(19): deoxycytidine
  modified_base (20)..(22): modified with 2′-O-methyl
  modified_base (20)..(22): modified with phosphorothioate
cuuacucuucuuuuctctcuua

SEQ ID NO: 17 - DNA, 14 bp, Artificial Sequence
  Synthetic
  modified_base (1)..(5): modified with 2′-O-methyl
  misc_feature (6)..(8): deoxythymine
  misc_feature (9)..(9): deoxyadensosine
  modified_base (10)..(14): modified with 2′-O-methyl
cagactttauucaa

SEQ ID NO: 18 - DNA, 22 bp, Artificial Sequence
  Synthetic
attctctcttttcttctcauuc

SEQ ID NO: 19 - DNA, 22 bp, Artificial Sequence
  Synthetic
attctctcttcccttctcaccc

SEQ ID NO: 20 - DNA, 16 bp, Artificial Sequence
  Synthetic
attctctctccccttc

SEQ ID NO: 21 - DNA, 22 bp, Artificial Sequence
  Synthetic
auuctctcuuuucttctcattc

SEQ ID NO: 22 - DNA, 22 bp, Artificial Sequence
  Synthetic
auuctctcuucccttctcaccc

SEQ ID NO: 23 - DNA, 16 bp, Artificial Sequence
  Synthetic
auuctctcuccccttc